(19) United States
(12) Patent Application Publication
(10) Pub. No.: US 2006/0025430 A1
(43) Pub. Date: Feb. 2, 2006

(54) Novel irreversible inhibitors of epidermal growth factor receptor tyrosine kinase and uses thereof for therapy and diagnosis

(76) Inventors: Eyal Mishani, Mevasseret Zion (IL); Yulia Rozen, Vancouver (CA); Galith Abourbeh, Jerusalem (IL); Alexander Levitzki, Jerusalem (IL)

Correspondence Address: Martin D. Moynihan;PTRSI, Inc., P.O. Box 16446, Arlington, VA, 22202, US

(21) Appl. No.: 11/185,698

(22) Filed: Jul. 21, 2005

Related U.S. Application Data
(63) Continuation-in-part of application No. PCT/IL04/00068, filed on Jan. 22, 2004.
(60) Provisional application No. 60/441,779, filed on Jan. 23, 2003.

(51) Int. Cl. A61K 31/517; C07D 239/84

(52) U.S. Cl. 514/266.1; 514/266.4; 544/293; 544/244

(57) ABSTRACT

Novel epidermal growth factor receptor tyrosine kinase (EGFR-TK) irreversible inhibitors, pharmaceutical compositions including same and their use in the treatment of EGFR-TK related diseases or disorders are disclosed. Novel radiolabeled EGFR-TK irreversible inhibitors as their use as biomarkers for medicinal radioimaging such as Positron Emission Tomography (PET) and Single Photon Emission Computed Tomography (SPECT) and as radiopharmaceuticals for radiotherapy are further disclosed.

### RELATED APPLICATIONS

This is a Continuation-In-Part (CIP) of PCT Application No. PCT/IL2004/000068, filed on Jan. 22, 2004, which claims the benefit under § 119(e) of U.S. Provisional Application No. 60/441,779, filed on Jan. 23, 2003.

### FIELD AND BACKGROUND OF THE INVENTION

The present invention relates to novel compounds and their use in therapy (e.g., cancer therapy) and diagnosis. More particularly, the present invention relates to novel irreversible inhibitors of epidermal growth factor receptor tyrosine kinase (EGFR-TK) and their use in the treatment of EGFR-TK related diseases and disorders (e.g., cancer), and to novel radiolabeled EGFR-TK irreversible inhibitors and their use as biomarkers for medicinal radioimaging such as Positron Emission Tomography (PET) and Single Photon Emission Computed Tomography (SPECT), and as radiopharmaceuticals for radiotherapy.

The presently used anticancer therapy is mostly based on non-specific cytotoxic agents, such as cisplatin, paclitaxel, doxorubicin, topotecan and 5-fluorouracil (5-FU). These cytotoxic agents are mainly directed to induce DNA damage, inhibit DNA synthesis or disrupt the cytoskeleton. The toxicity of these agents limits their dosage quantities, which often results in the disease recurrence. In some cases, the maximum tolerated dose is even below the minimum effective dose for tumor regression (Ciardiello, 2000; Renhowe, 2001; Rowinsky, 2000).

The realization that cancer cells differ from normal cells in their aberrant signal transduction has given impetus to cancer researchers to target the cancer cells while searching for cancer therapy and more recently for cancer diagnosis.

Polypeptides such as growth factors, differentiation factors, and hormones often mediate their pleiotropic actions by binding to and activating cell surface receptors with an intrinsic intracellular protein tyrosine kinase activity.

The epidermal growth factor receptor (EGFR, Erb-B1) belongs to a family of proteins, involved in the proliferation of normal and malignant cells (Artega et al., 2001). Overexpression of Epidermal Growth Factor Receptor (EGFR) is present in at least 70% of human cancers (Seymour, 2001) such as, non-small cell lung carcinomas (NSCLC), breast cancers, gliomas, squamous cell carcinoma of the head and neck, and prostate cancer (Raymond et al., 2000, Salomon et al., 1995, Voldborg et al., 1997). The EGFR is therefore widely recognized as an attractive target for the design and development of compounds that can specifically bind and inhibit the tyrosine kinase activity and its signal transduction pathway in cancer cells, and thus can serve as either diagnostic or therapeutic agents.

For example, the EGFR tyrosine kinase (EGFR-TK) reversible inhibitor, Iressa® (see, FIG. 1), was recently approved by the FDA for treatment of NSCLC and prostate cancer, and several other anti-EGFR targeted molecules, such as Tarceva® (FIG. 1) and the anti-EGFR antibody Erbitux®, are presently undergoing clinical Phase 3 trials. Consequently, there has been a growing interest in the use of EGFR-TK inhibitors as radiotracers for molecular imaging of EGFR overexpressing tumors by nuclear medicine modalities and as radiotracers for radiotherapy.

Compounds belonging to the 4-Anilinoquinazolines family, which are also referred to herein as 4-(phenylamino)quinazolines, have been shown to potently and selectively inhibit EGFR-TK activity by binding reversibly to an inner membrane ATP binding site on EGFR-TK, (Faaland et al., 1991; Miyaji et al., 1994; Gazit et al., 1996; Artega et al., 1997; Nelson and Fry, 1997; Johnstrom et al., 1997; Smaill et al., 1999; Tsou et al., 2001; and Han et al., 1996), the prototype for such compounds being the small-molecule AG 1478, also known as PD 153035 (Fry et al., 1994; Levitzki and Gazit, 1995), which is presently in clinical development. The FDA approved Iressa® described above also belongs to this quinazoline family (Baselga and Averbuch, 2000).

The potency of these reversible EGFR-TK inhibitors, however, is limited by their non-specific binding and rapid blood clearance, and thus, irreversible EGFR-TK inhibitors, which are based on the structure of AG 1478, have been proposed (Fry et al., 1998; Smaill et al., 2000; and U.S. Pat. Nos. 6,153,617 and 6,127,374). PD168393 and PD160678, which are representative examples of such irreversible inhibitors are presented in background art FIG. 1. The irreversible binding of these inhibitors was achieved by substituting the 6 or 7 position of the quinazoline ring of an 4-(anilino)quinazoline derivative with an α,β-unsaturated carboxylic group, preferably an acrylamide group, which binds covalently to the Cys-773 at the EGFR-TK ATP binding site. Some of these compounds showed high potency toward EGFR inhibition in both in vitro and in vivo experiments (Smaill et al., 2000). However, as is detailed hereinunder, more recent studies showed that these irreversible EGFR-TK inhibitors are limited by a relatively low accumulation at EGFR-expressing tumor cells.

Hence, it would be highly advantageous to have irreversible EGFR-TK inhibitors with improved efficacy, which could serve as potent anticancer agents. It would further be advantageous to have such irreversible EGFR-TK inhibitors that can be subjected to radiolabeling and thus could serve as potent radiopharmaceuticals and radioimaging agents.

The use of radioactive nuclides for medicinal purposes is well known in the art. Biologically active compounds that bind to specific cell surface receptors or that in other ways modify cellular functions have received some consideration as radiopharmaceuticals, and therefore, when labeled with a radioactive nuclide, such compounds are used as biospecific agents in radioimaging and radiotherapy.

Positron Emission Tomography (PET), a nuclear medicine imagine technology which allows the three-dimensional, quantitative determination of the distribution of radioactivity within the human body, is becoming an increasingly important tool for the measurement of physiological, biochemical, and pharmacological function at a molecular level, both in healthy and pathological states. PET requires the administration to a subject of a molecule labeled with a positron-emitting nuclide (radiotracer) such as ¹⁵O, ¹³N, ¹¹C, and ¹⁸F, which have half-lives of 2, 10, 20, and 110 minutes, respectively.

Single Photon Emission Computed Tomography (SPECT) is a form of chemical imaging in which emissions from radioactive compounds, labeled with gamma-emitting radionuclides, are used to create cross-sectional images of radioactivity distribution in vivo. SPECT requires the administration to a subject of a molecule labeled with a gamma-emitting nuclide such as ^{99m}Tc, ⁶⁷Ga, ¹¹¹In and ¹²³I.

The use of nuclear medicine imaging techniques such as Single Photon Emission Compute Tomography (SPECT) and Positron Emission Tomography (PET), along with a suitable radiotracer that binds to EGFR irreversibly, can therefore provide for in vivo drug development and identification of a lead chemical structure to be used as an EGFR-TK biospecific agent for radiotherapy or as a labeled bioprobe for diagnosis by radioimaging. Nuclear imaging can be further used for in vivo mapping and quantification of the receptor-kinase in cancer. Using a labeled EGFR-TK irreversible inhibitor would enable both the identification of patients having tumors overexpressing EGFR, and the study of changes in the levels of EGFR expression during therapy. Such a diagnostic method can lead to a better patient management and differentiation in regards to therapeutic course of action. Moreover, the increasing demand to incorporate diagnostic methods into clinical studies of EGFR-targeted therapies suggests a potential future use of EGFR-labeled inhibitors.

Radiolabeling of 4-anilinoquinazoline EGFR-TK inhibitors has been reported in the art. For example, a radioiodinated analog of PD 153035 and in vitro binding studies therewith in MDA-486 cells have been reported (Mulholland et al., 1995). PD 153035 labeled with carbon-11 in the 6,7-methoxy groups has been evaluated in rats implanted with human neuroblastoma xenografts (SH-SY5Y) but specific uptake was not determined in a blocking study (Johnstrom et al, 1998). PD 153035 was also labeled with carbon-11 specifically at the 7-methoxy position and biodistribution experiments were performed in normal mice, but uptake specificity could not be demonstrated as administration of an enzyme-blocking dose of PD 153035 caused an increase in tracer uptake in the tissues studied (Mulholland et al., 1997). The same abstract reported the labeling of the 7-(2-fluoroethoxy) PD 153035 analog with fluorine-18, but no biological experiments with this tracer were described.

U.S. Pat. No. 6,126,917 (to the present inventors), Mishani et al., 1999 and Bonasera et al., 2000, all teach reversible inhibitors of EGFR-TK of the 4-anilinoquinazoline family labeled with fluorine-18 on the aniline ring. These compounds were tested in vitro, in vivo and by PET image analysis. While some of these compounds showed effective (reversible) inhibition activity in vitro, they were found to be somewhat ineffective as tracers for the imaging of EGFR-TK in vivo due to kinetic factors such as kₒₙ and k_{off} and rapid blood clearance, as was further demonstrated by an animal PET comparative study between fluorine-18 FDG and these radiolabeled compounds. It is assumed that the discrepancy between the encouraging in vitro results and the discouraging in vivo results derives from the ATP competition at the compounds' binding site.

In order to eliminate this ATP binding competition and thus obtain a better specificity and inhibitory effect of radiolabeled EGFR-TK inhibitors, which would potentially result in higher diagnostic performance and high radiotherapeutic activity in tumor cells expressing EGFR-TK, radiolabeled irreversible inhibitors, based on those described by Smaill et al. (Smaill et al., 2000), were synthesized. As is taught in U.S. Pat. No. 6,562,319 (to the present inventors) and in Ben David et al., 2003, acrylamido derivatives of 4-anilinoquinazoline were synsthesized, radiolabeled by ¹¹C and were tested for PET imaging of tumor cells overexpressing EGFR-TK. Indeed, these compounds showed irreversible and fast binding effect toward EGFR in in vitro studies conducted with A431 cells. However, while the ATP binding competition was eliminated and long-term inhibitory effect was obtained with these compounds in vitro, the in vivo studies in tumor bearing rats did not indicate high accumulation of the compounds in the tumor. In further in vivo studies fast decomposition and clearance, as well as high accumulation of the compounds in the intestine, were observed, suggesting that the performance of this class of compounds is limited by low in vivo bioavailability and degradation.

There is thus a widely recognized need for, and it would be highly advantageous to have, novel irreversible inhibitors of EGFR-TK devoid of the above limitations, which can be further subjected to radiolabeling.

### SUMMARY OF THE INVENTION

According to the present invention there are provided novel compounds that are irreversible inhibitors of EGFR-TK and methods of using same in treating EGFR-TK related diseases and disorders. Further according to the present invention there are provided novel radiolabeled irreversible inhibitors of EGFR-TK and methods of using same in radioimaging and radiotherapy.

According to one aspect of the present invention, there is provided a compound having the general Formula I: 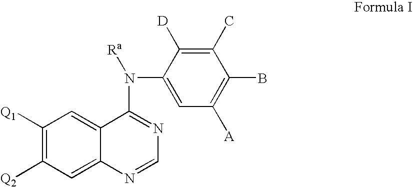
wherein:
Q1 is X—W(═Y)-Z and Q2 is selected from the group consisting of hydrogen, halogen, alkoxy, hydroxy, thiohydroxy, thioalkoxy, alkylamino and amino, or
Q1 is selected from the group consisting of hydrogen, halogen, alkoxy, hydroxy, thiohydroxy, thioalkoxy, alkylamino and amino and Q2 is X—W(═Y)-Z;
X is selected from the group consisting of —NR¹—, —O—, —NH—NR¹—, —O—NR¹—, NH—CHR¹—, —CHR¹—NH—, —CHR¹—O—, —O—CHR¹—, —CHR¹—CH₂— and —CHR¹—S— or absent;
W is carbon;
Y is selected from the group consisting of oxygen and sulfur;
Z is —CR²R³R⁴;
R^{a} is selected from the group consisting of hydrogen or alkyl having 1-8 carbon atoms;
A, B, C and D are each independently selected from the group consisting hydrogen and a first derivatizing group;
R¹ is selected from the group consisting of hydrogen, and substituted or non-substituted alkyl having 1-6 carbon atoms;
R² is a leaving group; and
R³ and R⁴ are each independently selected from the group consisting of hydrogen and a second derivatizing group.

According to further features in preferred embodiments of the invention described below, the first derivatizing group is selected from the group consisting of hydrogen, halogen, alkyl, haloalkyl, hydroxy, alkoxy, carboxy, carbalkoxy, thiocarboxy, thiohydroxy, thioalkoxy, sulfinyl, sulfonyl, amino, alkylamino, carbamyl, nitro and cyano.

According to still further features in the described preferred embodiments the second derivatizing group is selected from the group consisting of halogen, alkyl, haloalkyl, cycloalkyl, heteroalicyclic, aryl, heteroaryl, carboxy, hydroxy, alkoxy, aryloxy, carbonyl, thioalkoxy, thiohydroxy, thioaryloxy, thiocarboxy, thiocarbonyl, sulfinyl, sulfonyl, amino, alkylamino, carbamyl, nitro and cyano, or alternatively, R³ and R⁴ together form a five- or six-membered ring.

According to still further features in the described preferred embodiments the leaving group is selected from the group consisting of halogen, alkoxy, aryloxy, thioalkoxy, thioaryloxy, azide, sulfinyl, sulfonyl, sulfonamide, phosphonyl, phosphinyl, carboxy and carbamyl.

According to still further features in the described preferred embodiments the alkoxy comprises a morpholino group.

According to still further features in the described preferred embodiments the alkylamino comprises a N-piperazinyl group.

According to still further features in the described preferred embodiments the Q1 is X—W(═Y)-Z and Q2 is selected from the group consisting of hydrogen, halogen, alkoxy, hydroxy, thiohydroxy, thioalkoxy, alkylamino and amino. Preferably, Q2 is hydrogen, alkoxy or alkylamino, as described hereinabove. Further preferably, X is —NR¹— and Y is oxygen. Further preferably each of R¹, R³ and R⁴ is hydrogen. Further preferably, R² is a leaving group selected from the group consisting of alkoxy and halogen.

According to still further features in the described preferred embodiments at least one of A, B, C and D is fluorine. Preferably D is fluorine. More preferably, D is fluorine, A and B are each chlorine and C is hydrogen.

According to still further features in the described preferred embodiments A is bromine or iodine. Preferably, A is bromine or iodine and B, C and D are each hydrogen.

According to another aspect of the present invention, there is provided a pharmaceutical composition comprising as an active ingredient the compound described hereinabove and a pharmaceutical acceptable carrier.

The pharmaceutical composition can be packaged in a packaging material and identified in print, in or on the packaging material, for use in the treatment of an EGFR-tyrosine kinase related disease or disorder, such as a cell proliferative disorder.

The cell proliferative disorder can be, for example, papilloma, blastoglioma, Kaposi's sarcoma, melanoma, lung cancer, ovarian cancer, prostate cancer, squamous cell carcinoma, astrocytoma, head cancer, neck cancer, bladder cancer, breast cancer, lung cancer, colorectal cancer, thyroid cancer, pancreatic cancer, gastric cancer, hepatocellular carcinoma, leukemia, lymphoma, Hodgkin's disease and Burkitt's disease.

According to still another aspect of the present invention, there is provided a method of treating an EGFR-tyrosine kinase related disease or disorder, described hereinabove, in a subject in need thereof, which comprises administering to the subject a therapeutically effective amount of the pharmaceutical composition described hereinabove.

According to yet another aspect of the present invention, there is provided a method of inhibiting cell proliferation, which comprises subjecting the cell to the compound of the present invention, described hereinabove.

According to an additional aspect of the present invention, there is provided a method of synthesizing the compound of the present invention, which comprises: (a) coupling an aniline derivatized by R^{a}, A, B, C, and D, as described hereinabove, with a 4-chloroquinazoline substituted at position 6 and/or 7 by at least one reactive group, so as to produce a reactive 4-(phenylamino)quinazoline derivatized by A, B, C and D; and (b) reacting the reactive 4-(phenylamino)quinazoline with a reactive carboxylic derivative substituted at the a position by R², R³ and R⁴, as described hereinabove.

In cases where the reactive 4-(phenylamino)quinazoline is 4-(phenylamino)-6-nitroquinazoline, the method further comprises, prior to step (b): (c) reducing the 4-(phenylamino)-6-nitroquinazoline so as to produce a 4-(phenylamino)-6-aminoquinazoline derivatized by A, B, C and D.

When the 4-chloroquinazoline is substituted at positions 6 and 7 by a first and a second reactive groups, the method can further comprise, prior to step (b): (d) reacting the reactive 4-(phenylamino)quinazoline with a chemically reactive group, such as, for example, a morpholinoalkoxy group or a N-piperazinyl group.

The reactive carboxylic derivative is preferably selected from the group consisting of α-chloroacetyl chloride and α-methoxyacetyl chloride.

The compounds described hereinabove can be radiolabeled by various radioisotopes. Hence, according to yet an additional aspect of the present invention there is provided a radiolabeled compound having the general Formula described hereinabove, wherein:
Q1 is X—W(═Y)-Z and Q2 is selected from the group consisting of hydrogen, halogen, alkoxy, hydroxy, thiohydroxy, thioalkoxy, alkylamino and amino, or
Q1 is selected from the group consisting of hydrogen, halogen, alkoxy, hydroxy, thiohydroxy, thioalkoxy, alkylamino and amino and Q2 is X—W(═Y)-Z;
X is selected from the group consisting of —NR¹—, —O—, —NH—NR¹—, —O—NR¹—, NH—CHR¹—, —CHR¹—NH—, —CHR¹—O—, —O—CHR¹—, —CHR¹—CH₂— and —CHR¹—S— or absent;
W is carbon;
Y is selected from the group consisting of oxygen and sulfur;
Z is —CR²R³R⁴;
R^{a} is selected from the group consisting of hydrogen or alkyl having 1-8 carbon atoms;
A, B, C and D are each independently selected from the group consisting of hydrogen, a first non-radioactive derivatizing group and a first radioactive derivatizing group selected from a radioactive bromine, a radioactive iodine and a radioactive fluorine;
R¹ is selected from the group consisting of hydrogen, and substituted or non-substituted alkyl having 1-6 carbon atoms;
R² is a leaving group; and
R³ and R⁴ are each independently selected from the group consisting of hydrogen, a second non-radioactive derivatizing group and a second radioactive derivatizing group containing a radioactive carbon, a radioactive fluorine, a radioactive bromine and/or a radioactive iodine; provided that the compound comprises at least one radioactive atom.

Preferred radiolabeled compounds according to the present invention include the preferred compounds described hereinabove, having one or more radioactive atoms as follows:

In one embodiment, at least one of A, B, C and D is a radioactive fluorine. Preferably D is a radioactive fluorine. More preferably, D is a radioactive fluorine, A and B are each chlorine and C is hydrogen.

In another embodiment, A is a radioactive bromine or a radioactive iodine.

Hence, according to further features in preferred embodiments of the invention described below, at least one of A, B, C and D is a radioactive atom selected from the group consisting of a radioactive fluorine, a radioactive bromine and a radioactive iodine.

According to still further features in the described preferred embodiments the radioactive fluorine is fluorine-18, the radioactive bromine is bromine-76 or bromine-77, the radioactive iodine is iodine-123, iodine-124 or iodine-131, preferably iodine-124, and the radioactive carbon is carbon-111.

According to still an additional aspect of the present invention, there is provided a pharmaceutical composition comprising as an active ingredient the radiolabeled compound of the present invention, as described hereinabove, and a pharmaceutical acceptable carrier.

According to a further aspect of the present invention there is provided a method of monitoring the level of epidermal growth factor receptor within a body of a patient, which comprises: (a) administering to the patient the radiolabeled compound of the present invention; and (b) employing a nuclear imaging technique for monitoring a distribution of the compound within the body or within a portion thereof.

The technique is preferably positron emission tomography or single photon emission computed tomography.

The radioactive atom is preferably a radioactive iodine, a radioactive bromine or a radioactive fluorine.

According to yet a further aspect of the present invention there is provided a method of radiotherapy, comprising administering to a patient a therapeutically effective amount of the radiolabeled compound of the present invention.

The radioactive atom is preferably a radioactive iodine or a radioactive bromine.

According to further aspects of the present invention there are provided methods of synthesizing the radiolabeled compounds described hereinabove.

For compounds in which at least one of A, B, C and D is fluorine-18, the method comprises: (a) providing a fluorine-18 labeled aniline derivatized by the R^{a}, A, B, C and D, wherein at least one of A, B, C and D is the fluorine-18; (b) coupling the fluorine-18 labeled aniline derivatized by the R^{a}, A, B, C and D with 4-chloroquinazoline substituted at position 6 and/or 7 by at least one reactive group, so as to produce a reactive fluorine-18 labeled 4-(phenylamino)quinazoline derivatized by the A, B, C and D; and (c) reacting the reactive fluorine-18 labeled 4-(phenylamino)quinazoline with a reactive carboxylic derivative substituted at the α position by the R², R³ and R⁴.

Alternatively, the method comprises:
(a) coupling an aniline derivatized by amine, by the R^{a}, and by three of the A, B, C and D which are not the fluorine-18, with a 4-chloroquinazoline substituted at position 6 or 7 by a first reactive group, so as to produce a reactive 4-(amino-substituted phenylamino) quinazoline derivatized by the amine, the R^{a}, and three of the A, B, C and D which are not the fluorine-18;
(b) converting the reactive 4-(amino-substituted phenylamino)quinazoline derivatized by the amine, the R^{a}, and three of the A, B, C and D which are not the fluorine-18 into a quaternary ammonium salt thereof;
(c) reacting the quaternary ammonium salt with a fluorine-18 labeled ion, so as to produce a reactive fluorine-18 labeled 4-(phenylamino)quinazoline derivatized by the R^{a}, A, B, C and D; and
(d) reacting the reactive fluorine-18 labeled 4-(phenylamino)quinazoline with a reactive carboxylic derivative substituted at the a position by the R², R³ and R⁴.

For compounds in which at least one of A, B, C and D is the radioactive bromine or the radioactive iodine, the method comprises: (a) coupling an aniline derivatized by the R^{a}, A, B, C and D, wherein at least one of A, B, C and D is a halogen, with a 4-chloroquinazoline substituted at position 6 and/or 7 by at least one reactive group, so as to produce a reactive 4-(phenylamino)quinazoline derivatized by the A, B, C and D, wherein at least one of A, B, C and D is the halogen; (b) radiolabeling the reactive 4-(phenylamino)quinazoline derivatized by the A, B, C and D with a radioactive bromine or a radioactive iodine, so as to produce a radioactive bromine labeled or a radioactive iodine labeled reactive 4-(phenylamino)quinazoline derivatized by the A, B, C and D, wherein at least one of the A, B, C and D is the radioactive bromine or the radioactive iodine; and (c) reacting the radioactive bromine labeled or radioactive iodine labeled reactive 4-(phenylamino)quinazoline with a reactive carboxylic derivative substituted at the α position by the R², R³ and R⁴. The halogen is preferably bromine.

For compounds in which at least one of R³ and R⁴ is a second radioactive derivatizing group containing a radioactive fluorine, a radioactive bromine, a radioactive iodine and/or a radioactive iodine, the method comprises: (a) coupling an aniline derivatized by the R^{a}, A, B, C and D with a 4-chloroquinazoline substituted at position 6 and/or 7 by at least one reactive group, so as to produce a reactive 4-(phenylamino)quinazoline derivatized by the A, B, C and D; and (b) reacting the reactive 4-(phenylamino)quinazoline with a radiolabeled reactive carboxylic derivative substituted at the α position by the R², R³ and R⁴.

In each of the methods described above, the reactive carboxylic derivative is preferably selected from the group consisting of α-chloroacetyl chloride and α-methoxyacetyl chloride.

Each of the methods described above can further comprise reducing the 4-(phenylamino)-6-nitroquinazoline (non-labeled or fluorine-18 labeled), so as to produce the corresponding 4-(phenylamino)-6-aminoquinazoline.

In cases where the 4-chloroquinazoline is substituted at positions 6 and 7 by a first and a second reactive groups, each of the methods described above can further comprise reacting the reactive fluorine-18 labeled 4-(phenylamino)quinazoline with a chemically reactive group (e.g., a morpholinoalkoxy group or a N-piperazinyl group).

The present invention successfully addresses the shortcomings of the presently known configurations by providing novel irreversible EGFR-TK inhibitors with improved biostability and bioavailability, which can therefore be efficiently used as therapeutic agents and which can further be radiolabeled and thus serve as biomarkers for radioimaging and as radiopharmaceuticals for radiotherapy.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 presents background art chemical structures of reversible (Irresa and Terceva, FIG. 1a) and irreversible (PD 168393 and PD 160678, FIG. 1b) EGFR inhibitors;

FIG. 2 is a scheme presenting the synthetic route for preparing representative examples of irreversible EGFR-TK inhibitors according to the present invention, (Compounds 1-6);

FIG. 3 is a scheme presenting a representative radiosynthetic route for preparing representative examples of fluorine-18 labeled irreversible EGFR-TK inhibitors according to the present invention (fluorine-18 labeled Compounds 5 and 6);

FIG. 4 is a scheme presenting a representative radiosynthetic route for preparing representative examples of radioactive bromine and radioactive iodine labeled irreversible EGFR-TK inhibitors according to the present invention (radioactive bromine labeled Compounds 1 and 2 and radioactive iodine labeled Compounds 3 and 4);

FIG. 5 presents a bar graph showing the EGFR autophosphorylation level in A431 cells following incubation with various concentrations of Compound 5 and EGF stimulation-lysis after 1 hour incubation (filled bars) and following 8 hours post-incubation (bars with squared pattern);

FIG. 6 present Western Blots showing the EGFR autophosphorylation level in A431 cells following incubation with various concentrations of Compound 6 (upper row) and Compound 5 (bottom row) and EGF stimulation-lysis after 1 hour incubation (left) and following 8 hours post-incubation (right);

FIG. 7 presents comparative plots showing the reaction profile of Compound 5 with reduced glutathione, compared with that of {4-[(3,4-dichloro-6-fluorophenyl)amino]quinazoline-6-yl}acrylamide; and

FIGS. 8a-b presents plots showing the reaction rate of Compound 5 with reduced gluthatione as a function of the temperature (FIG. 8a) and the calculation of of the activation energy thereof (FIG. 8b).

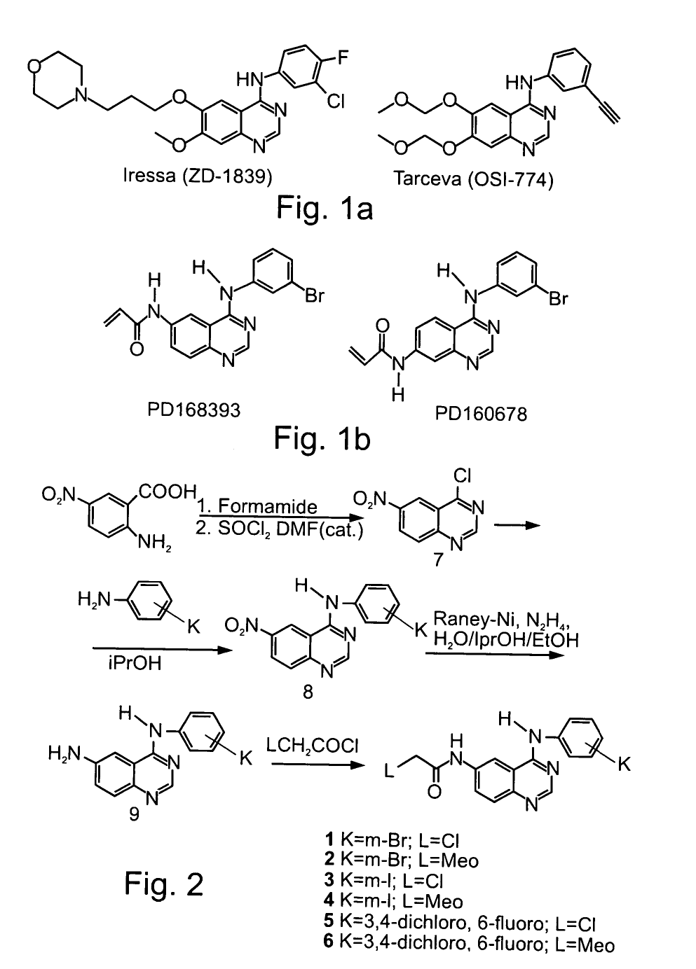

### DESCRIPTION OF THE PREFERRED EMBODIMENTS

The present invention is of novel compounds which are irreversible EGFR-TK inhibitors and can therefore be used in the treatment of EGFR related diseases or disorders, and which can further be radiolabeled and thus used as biomarkers for radioimaging such as Positron Emission Tomography (PET) and Single Photon Emission Computed Tomography (SPECT) and as radiopharmaceuticals for radiotherapy. Specifically, the non-labeled and radiolabeled compounds of the present invention can be used as therapeutic agents in the treatment of disorders or diseases, such as a variety of cancers, in which amplification, mutation and/or over expression of EGFR-TK has occurred, whereby the radiolabeled compounds of the present invention can be further used as irreversible PET or SPECT biomarkers for quantification, mapping and radiotherapy of such EGFR-TK associated diseases or disorders. The present invention is further of pharmaceutical compositions containing these compounds and of chemical and radio syntheses of these compounds.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the examples. The invention is capable of other embodiments or of being practiced or carried out in various ways. Also, it is to be understood that the phraseology and terminology employed herein is for the purpose of description and should not be regarded as limiting.

As is discussed in detail hereinabove, a novel class of 4-(phenylamino)quinazoline, which acts as irreversible EGFR-TK inhibitors has recently been uncovered. This class of compounds is characterized by a carboxylic moiety attached to the quinazoline ring, which includes an α,β-unsaturated side chain. The α,β-unsaturated side chain acts as a Michael acceptor that covalently binds to the Cys-773 at the EGFR-TK ATP binding site, and thus renders the inhibitor irreversible. However, while some of these compounds showed high potency toward EGFR inhibition in both in vitro and in vivo experiments (Smaill et al., 2000), the use of these compounds in applications such as nuclear imaging, in which high accumulation at EGFR-expressing tumor cells, bioavailability and reduced biodegradation are required, was found to be limited.

In a search for EGFR-TK irreversible inhibitors with improved in vivo performance, the present inventors have hypothesized that modifying certain structural and chemical features of the irreversible inhibitors described above such that the chemical reactivity thereof would be reduced without affecting their irreversible binding nature, would result in irreversible inhibitors with reduced biodegradation, enhanced bioavailability and thus with the required in vivo performance for both diagnostic and therapeutic applications. More specifically, it was envisioned that replacing the α,β-unsaturated side chain of the carboxylic moiety, which is a highly chemical reactive group, by a less reactive group, would enhance the biostability of the inhibitor. It was further envisioned that replacement of the α,β-unsaturated side chain by a leaving group would result in a side chain in which the α carbon to the carboxylic moiety is partially positively charged and thus sufficiently susceptible to a nucleophilic attack by the cystein moiety at the receptor binding site, and would therefore lead to a covalent bond formation therebetween, such that the irreversible nature of such an inhibitor would not be affected. However, it was further hypothesized that since the energy gaps of the HOMO LUMO electronic orbitals of such a α carbon center are higher than those of the β carbon in the α,β-unsaturated group, the bioavailability of such compounds would be increased, as compared with the acrylamide derivative. In view of the above, it was further assumed that if the inhibitory potency of such compounds will not be dramatically affected by the proposed structural change depicted above, such that the effective amount thereof will remain in the nanomolar range (as that of the presently known irreversible EGFR-TK inhibitors), these inhibitors would be retained at the receptor binding site long enough so as to allow covalent bonding, and thus may act as efficient irreversible EGFR-TK inhibitors characterized by enhanced bioavailability and biostability.

While reducing the present invention to practice, it was indeed found that such newly designed compounds, having an α-chloroacetamide or an α-methoxyacetamide group attached to the quinazoline ring, show high affinity toward EGFR and high ability to irreversibly bind to the receptor, thus indicating their potential as improved EGFR-TK irreversible inhibitors and as a result as improved therapeutic agents. It was further found that by designing such compounds that could be further subjected to radiolabeling by various radioisotopes, novel radiolabeled EGFR-TK irreversible inhibitors, which can serve as improved diagnostic and radiotherapeutic agents, were prepared.

Thus, according to one aspect of the present invention there is provided a compound having the general Formula I: 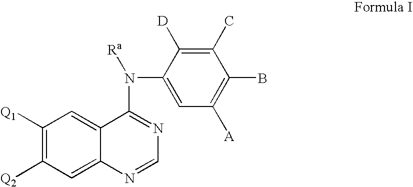
wherein:
Q1 is X—W(═Y)-Z and Q2 is selected from the group consisting of hydrogen, halogen, alkoxy, hydroxy, thiohydroxy, thioalkoxy, alkylamino and amino, or Q1 is selected from the group consisting of hydrogen, halogen, alkoxy, hydroxy, thiohydroxy, thioalkoxy, alkylamino and amino and Q2 is X—W(═Y)-Z;
X is selected from the group consisting of —NR¹—, —O—, —NH—NR¹—, —O—NR¹—, NH—CHR¹—, —CHR¹—NH—, —CHR¹—O—, —O—CHR¹—, —CHR¹—CH₂— and —CHR¹—S— or absent;
W is carbon;
Y is selected from the group consisting of oxygen and sulfur;
Z is —CR²R³R⁴;
R^{a} is selected from the group consisting of hydrogen or alkyl having 1-8 carbon atoms;
A, B, C and D are each independently selected from the group consisting hydrogen and a first derivatizing group;
R¹ is selected from the group consisting of hydrogen, and substituted or non-substituted alkyl having 1-6 carbon atoms;
R² is a leaving group; and
R³ and R⁴ are each independently selected from the group consisting of hydrogen and a second derivatizing group.

As used herein, the phrase “derivatizing group” refers to a major portion of a group which is covalently attached to another group.

The term “halogen”, which is also referred to herein as “halo”, refers to fluorine, chlorine, bromine or iodine.

As used herein, the term “hydroxy” refers to an —OH group.

As used herein, the term “alkyl” refers to a saturated aliphatic hydrocarbon including straight chain and branched chain groups. Preferably, the alkyl group is a medium size alkyl having 1 to 10 carbon atoms. More preferably, it is a lower alkyl having 1 to 6 carbon atoms. Most preferably it is an alkyl having 1 to 4 carbon atoms. Representative examples of an alkyl group are methyl, ethyl, propyl, isopropyl, butyl, tert-butyl, pentyl and hexyl.

The alkyl group, according to the present invention, may be substituted or non-substituted. When substituted, the substituent group can be, for example, cycloalkyl, alkenyl, aryl, heteroaryl, heteroalicyclic, hydroxy, alkoxy, aryloxy, thiohydroxy, thioalkoxy, thioaryloxy, halo, perhalo, trihalomethyl, carboxy, alkoxycarbonyl, thiocarboxy, carbamyl, cyano, nitro, N-piperidinyl, N-piperazinyl, N₁-piperazinyl-N-4-alkyl, N-pyrrolidyl, pyridinyl, N-imidazoyl, N-morpholino, N-thiomorpholino, N-hexahydroazepine, amino or NRbRc, wherein Rb and Rc are each independently hydrogen, alkyl, hydroxyalkyl, cycloakyl, aryl, N-piperidinyl, N-piperazinyl, N₁-piperazinyl-N₄-alkyl, N-pyrrolidyl, pyridinyl, N-imidazoyl, N-morpholino, N-thiomorpholino and N-hexahydroazepine, as these terms are defined herein.

The term “haloalkyl” refers to an alkyl group, as defined hereinabove, which is substituted by one or more halogen atoms.

As used herein, the term “cycloalkyl” refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene and adamantane.

The term “alkoxy” refers to both an -O-alkyl and an -O-cycloalkyl group, as defined hereinabove. Representative examples of alkoxy groups include methoxy, ethoxy, propoxy and tert-butoxy.

The -O-alkyl and the O-cycloalkyl groups, according to the present invention, may be substituted or non-substituted. When substituted, the substituent group can be, for example, cycloalkyl, alkenyl, aryl, heteroaryl, heteroalicyclic, hydroxy, alkoxy, aryloxy, thiohydroxy, thioalkoxy, thioaryloxy, halo, perhalo, trihalomethyl, carboxy, alkoxycarbonyl, thiocarboxy, carbamyl, cyano, nitro, N-piperidinyl, N-piperazinyl, N₁-piperazinyl-N₄-alkyl, N-pyrrolidyl, pyridinyl, N-imidazoyl, N-morpholino, N-thiomorpholino, N-hexahydroazepine, amino or NRbRc, wherein Rb and Rc are each independently hydrogen, alkyl, hydroxyalkyl, N-piperidinyl, N-piperazinyl, N₁-piperazinyl-N₄-alkyl, N-pyrrolidyl, pyridinyl, N-imidazoyl, N-morpholino, N-thiomorpholino and N-hexahydroazepine, as these terms are defined herein.

The term “thiohydroxy” refers to a —SH group.

The term “thioalkoxy” refers to both an —S-alkyl group, and an —S-cycloalkyl group, as defined herein.

The term “amino” refers to a —NH₂ group.

The term “alkylamino” refers to a —NRbRc group wherein Rb and Rc are each independently hydrogen, alkyl, hydroxyalkyl, N-piperidinyl, N-piperazinyl, N₁-piperazinyl-N₄-alkyl, N-pyrrolidyl, pyridinyl, N-imidazoyl, N-morpholino, N-thiomorpholino and N-hexahydroazepine, as these terms are defined herein, or, alternatively, Rb and Rc are covalently attached one to the other so as to form a cyclic amino compound such as, but not limited to, N-piperidinyl, N-piperazinyl, N₁-piperazinyl-N-4-alkyl, N-pyrrolidyl, pyridinyl, N-imidazoyl, N-morpholino, N-thiomorpholino and N-hexahydroazepine.

The term “carboxy” refers to a —C(═O)—OR′ group, where R′ is hydrogen, alkyl, cycloalkyl, alkenyl, aryl, heteroaryl (bonded through a ring carbon) or heteroalicyclic (bonded through a ring carbon) as defined herein.

The term “alkoxycarbonyl”, which is also referred to herein interchangeably as “carbalkoxy”, refers to a carboxy group, as defined hereinabove, where R′ is not hydrogen.

The term “carbonyl” refers to a —C(═O)—R′ group, where R′ is as defined hereinabove.

The term “thiocarbonyl” refers to a —C(═S)—R′ group, where R′ is as defined hereinabove.

An “aryl” group refers to an all-carbon monocyclic or fused-ring polycyclic (i.e., rings which share adjacent pairs of carbon atoms) group having a completely conjugated pi-electron system. Examples, without limitation, of aryl groups are phenyl, naphthalenyl and anthracenyl.

A “phenyl” group, according to the present invention can be substituted by one to three substituents or non-substituted. When substituted, the substituent group may be, for example, halogen, alkyl, alkoxy, nitro, cyano, trihalomethyl, alkylamino or monocyclic heteroaryl.

The term “heteroaryl” group includes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system.

An “aryloxy” group refers to both an -O-aryl and an -O-heteroaryl group, as defined herein.

A “thioaryloxy” group refers to both an -S-aryl and an -S-heteroaryl group, as defined herein.

A “trihalomethyl” group refers to a —CX₃ group, wherein X is a halogen as defined herein. A representative example of a trihalomethyl group is a —CF₃ group.

A “perhalo” group refers to a group in which all the hydrogen atoms thereof have been replaced by halogen atoms.

A “thiocarboxy” group refers to a —C(═S)—OR′ group, where R′ is as defined herein.

A “sulfinyl” group refers to an —S(═O)—R′ group, where R′ is as defined herein.

A “sulfonyl” group refers to an —S(═O)₂—R′ group, where R′ is as defined herein.

A “carbamyl” group refers to an —OC(═O)—NRbRc group, where Rb and Rc are as defined herein.

A “nitro” group refers to a —NO₂ group.

A “cyano” group refers to a —C≡N group.

The term “N-piperazinyl”, which is also referred to herein as “N-piperazino”refers to a 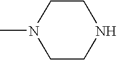
group.

The term “N-piperidinyl” refers to a 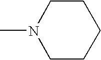
group.

The term “N₁-piperazinyl-N₄-alkyl” refers to a 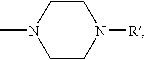
where R′ is an alkyl, as defined hereinabove.

The term “N-pyrrolidyl” refers to a 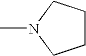
group.

The term “pyridinyl” refers to a 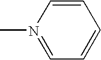
group.

The term “N-imidazoyl” refers to a 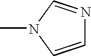
group.

The term “N-morpholino” refers to a 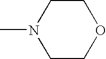
group.

The term “N-thiomorpholino” refers to a 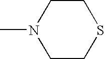
group.

The term “N-hexahydroazepine” refers to a 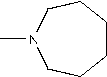
group.

The compounds of the present invention are therefore derivatized 4-(phenylamino)quinazolines, substituted at position 6 or 7 of the quinazoline ring by a carboxylic group that is substituted at the α position by a leaving group, which is also defined herein as a X—W(═Y)-Z group.

As used herein throughout, and is well known in the art, the phrase “leaving group” refers to a chemical moiety that can be easily replaced by a nucleophilic moiety in a nucleophilic reaction. Representative examples of leaving groups include, without limitation, halogen, alkoxy, aryloxy, thioalkoxy, thioaryloxy, sulfinyl, sulfonyl, carboxy and carbamyl, as these terms are defined hereinabove, with halogen and alkoxy being the presently most preferred. Additional examples of leaving groups include, without limitation, azide, sulfonamide, phosphonyl and phosphinyl.

As used herein, the term “azide” refers to a —N₃ group.

The term “sulfonamide” refers to a —S(═O)₂—NR′R″ group, with R′ as defined hereinabove and R″ as defined herein for R′.

The term “phosphonyl” describes an —O—P(═O)(OR′)₂ group, with R′ as defined hereinabove.

The term “phosphinyl” describes a —PR′R″ group, with R′ and R″ as defined hereinabove.

As is described in the art (see, for example, U.S. Pat. No. 6,126,917 and Smaill et al., 2000), the level of the biological activity of 4-(phenylamino)quinazoline EGFR-TK inhibitors, whether reversible or irreversible, is influenced by the nature of the derivatizing groups at both the anilino ring and the quinazoline ring thereof. The nature of these derivatizing groups may affect the binding affinity of the compound to the receptor as well as other biological activity parameters such as specificity, metabolism of the compound and kinetic rates.

Thus, according to a preferred embodiment of the present invention, the derivatizing group of the compound of the present invention is attached to the aniline ring (as is represented in Formula I hereinabove by A, B, C and D as a first derivatizing group) and includes, for example, hydrogen, halogen, alkyl, haloalkyl, hydroxy, alkoxy, carboxy, carbalkoxy, thiohydroxy, thiocarboxy, thioalkoxy, sulfinyl, sulfonyl, amino, alkylamino, carbamyl, nitro and cyano, as these terms are defined hereinabove.

According to another preferred embodiment of the invention, a derivatizing group is attached to the quinazoline group (as is represented in Formula I hereinabove by either Q1 or Q2) and includes, for example, halogen, alkoxy, hydroxy, thiohydroxy, thioalkoxy, alkylamino and amino. Preferably, this derivatizing group is an alkoxy group and, more preferably, it is an alkoxy group that comprises a morpholino group such as, but not limited to, a 3-(4-morpholinyl)propoxy group. Further preferably, the derivatizing group is a substituted or non-substituted morpholino group or a substituted or non-substituted piperazino group. The presence of a morpholino or piperazino group in this class of compounds in known to increase their biological availability (Smaill et al., 2000).

Another factor which influences the binding potency of the compounds of the present invention is the position of which the carboxylic group is attached to the quinazoline ring. A 6-position carboxylic group has higher binding potency to the EGFR-TK ATP site (Smaill et al, 1999, Smaill et al., 2000 and U.S. Pat. Nos. 6,153,617 and 6,127,374). Thus, according to another preferred embodiment of the present invention, the X—W(═Y)-Z group of the compound is attached to position 6 of the quinazoline ring, such that Q1 in Formula I above is X—W(═Y)-Z.

According to still another preferred embodiment of the invention, the 6-position carboxylic group substituted by a leaving group is an α-chloroacetamide or α-methoxyacetamide group. Thus, preferred compounds according to the present invention are N-[4-(phenylamino)quinazolin-6-yl]-2-chloroacetamide and N-[4-(phenylamino)quinazolin-6-yl]-2-methoxyacetamide, derivatized by the R^{a}, A, B, C and D as these symbols are defined above, with the first being more active and therefore presently more preferred. These compounds are represented by Formula I hereinabove, wherein Q1 is X—W(═Y)-Z, X is —NH—, Y is oxygen, and Z is —CH₂C₁ or CH₂OCH₃, respectively.

As is taught, for example, in U.S. Pat. No. 6,126,917, 4-(phenylamino)quinazolines that are derivatized at position 6 of the anilino group by fluorine are potent inhibitors of EGFR-TK. The highest affinity toward the receptor is achieved using 4-[(3,4-dichloro-6-fluorophenyl)-amino]quinazolines.

Thus, preferred compounds according to the present invention are those in which R^{a} is hydrogen, A and B are each chlorine, C is hydrogen and D is fluorine. More preferred compounds are the N-[4-(phenylamino)quinazolin-6-yl]-2-chloroacetamide and N-[4-(phenylamino)quinazolin-6-yl]-2-methoxyacetamide described hereinabove, in which R^{a} is hydrogen, A and B are each chlorine, C is hydrogen and D is fluorine. These compounds are referred to hereinbelow as Compound 5 and compound 6, respectively.

As is taught in U.S. Pat. No. 6,562,319 and in U.S. Application No. 20020128553, 4-(phenylamino)quinazolines that are derivatized at position 3 of the anilino group by bromine or iodine are also potent inhibitors of EGFR-TK. These compounds further serve as precursors for radioactive bromine or radioactive iodine labeled compounds, which, as is detailed hereinbelow, are highly potent radiolabeled compounds.

Hence, additional preferred compounds according to the present invention are those in which R^{a} is hydrogen, A is bromine or iodine and B, C and D are each hydrogen. More preferred compounds are the N-[4-(phenylamino)quinazolin-6-yl]-2-chloroacetamide and N-[4-(phenylamino)quinazolin-6-yl]-2-methoxyacetamide described hereinabove, in which R^{a} is hydrogen, is bromine or iodine and B, C and D are each hydrogen. These compounds are referred to hereinbelow as Compounds 1-4.

As is discussed hereinabove, each of the preferred compounds described above may be further advantageously derivatized by an alkoxy (e.g., a 3-(4-morpholinyl)propoxy group) or an alkylamino group (e.g., a piperazino group) at position 7 of the quinazoline ring.

The carboxylic group substituted by a leaving group (represented by X—W(═Y)-Z in Formula I hereinabove) can be further substituted by one or more derivatizing groups (as is represented in Formula I hereinabove by R³ and/or R⁴ as a second derivatizing group). Such derivatizing groups can be, for example, halogen, alkyl, haloalkyl, cycloalkyl, heteroalicyclic, aryl, heteroaryl, carboxy, hydroxy, alkoxy, aryloxy, carbonyl, thioalkoxy, thiohydroxy, thioaryloxy, thiocarboxy, thiocarbonyl, sulfinyl, sulfonyl, amino, alkylamino, carbamyl, nitro and cyano, as these terms are defined hereinabove. Alternatively, R³ and R⁴ can together form a five- or six-membered ring, such as, for example, cycloalkyl, heteroalicyclic, phenyl or heteroaryl, as these terms are defined hereinabove.

Chemical Syntheses:

According to another aspect of the present invention, there is provided a method for synthesizing the compounds of the invention. The method is effected by coupling an aniline derivatized by the R^{a}, A, B, C and D described hereinabove with a 4-chloroquinazoline substituted at position 6 and/or 7 by one or more reactive group(s), so as to produce a reactive 4-(phenylamino)quinazoline derivatized by R^{a}, A, B, C and D, and reacting the reactive 4-(phenylamino)quinazoline with a reactive carboxylic derivative substituted at the a position by a leaving group, and optionally by a derivatizing group, as is described hereinabove. Alternatively, the method further includes reacting the reactive 4-(phenylamino)quinazoline with a chemically reactive group, prior to its reaction with the reactive carboxylic derivative, so as to produce a reactive substituted 4-(phenylamino)quinazoline.

As used herein, the term “reactive” with respect to a group or a derivative refers to a group or derivative which can be easily reacted with another group so as to produce a new compound that comprises a new functional group. Representative examples of a reactive group include nitro, amino, hydroxy, alkoxy and halogen. A carboxylic acid chloride is a representative example of a reactive carboxylic derivative. An alkoxy group which comprises a metal salt of hydroxyalkyl is a representative example of a chemically reactive group. Preferably, the chemically reactive group comprises a metal salt, e.g., sodium salt, potassium salt or lithium salt, of 3-(4-morpholinyl)-1-propanol, which is also referred to herein as 3-(4-morpholinyl)propoxy.

In one particular, which includes a quinazoline that is substituted by one reactive group at position 6 thereof, 3,4-dichloro-6-fluoroaniline is reacted with 4-chloro-6-nitroquinazoline, so as to produce 4-[(3,4-dichloro-6-fluorophenyl)amino]-6-nitroquinazoline, which is reduced, by means of an ethanolic solution of hydrazine hydrate and Raney®Nickel, so as to produce 4-[(3,4-dichloro-6-fluorophenyl)amino]-6-aminoquinazoline. Then, the 4-[(3,4-dichloro-6-fluorophenyl)amino]-6-aminoquinazoline is reacted with α-chloroacetyl chloride or α-methoxyacetyl chloride, so as to produce N-{4-[(3,4-dichloro-6-fluorophenyl)amino]quinazoline-6-yl}-2-chloroacetamide (Compound 5) and N-{4-[(3,4-dichloro-6-fluorophenyl) amino]quinazoline-6-yl}-2-methoxycetamide, respectively (Compound 6).

In another particular, the starting material is 3-bromoaniline and the final product is N-{4-[(3-bromophenyl)amino]quinazoline-6-yl}-2-chloroacetamide (Compound 1) or N-{4-[(3-bromophenyl)amino]quinazoline-6-yl}}-2-methoxyacetamide (Compound 2).

In still another particular, the starting material is 3-iodoaniline and the final product is N-{4-[(3-iodophenyl)amino]quinazoline-6-yl}-2-chloroacetamide (Compound 3) or N-{4-[(3-iodophenyl)amino]quinazoline-6-yl}}-2-methoxyacetamide (Compound 4).

In yet another particular, which includes a quinazoline that is substituted by two different reactive groups at positions 6 and 7 thereof, any of the derivatized anilines described above is reacted with 4-chloro-7-fluoro-6-nitroquinazoline, so as to produce a derivatized 4-[(phenyl)amino]-7-fluoro-6-nitroquinazoline. The derivatized 4-[(phenyl)amino]-7-fluoro-6-nitroquinazoline is then reacted with a sodium salt of 3-(4-morpholinyl-1-propanol), so as to produce a derivatized 4-[(phenyl)amino]-7-[3-(4-morpholinyl)propoxy]-6-nitroquinazoline, which is reduced, by means of an ethanolic solution of hydrazine hydrate and Raney®Nickel, so as to produce a derivatized 6-amino-4-[(phenyl)amino]-7-[3-(4-morpholinyl)propoxy]quinazoline. The product is then reacted with 2-chloroacetyl chloride or 2-methoxyacetyl chloride, so as to produce a morpholino-substituted compound according to the present invention.

Alternatively, the derivatized 4-[(phenyl)amino]-7-fluoro-6-nitroquinazoline can be similarly reacted with a sodium salt of piperazinyl, so as to produce a piperazinyl-substituted compound according to the present invention.

The Biochemistry:

As is demonstrated in Examples section that follows, representative examples of the novel compounds of the present invention were tested for their binding to EGFR and showed high affinity toward EGFR and substantial irreversible binding thereto. These compounds can therefore efficiently serve for treating diseases or disorders in which inhibiting the activity of EGFR-TK is beneficial.

Hence, according to another aspect of the present invention, there is provided a method of treating an EGFR-TK related disease or disorder. The method according to this aspect of the present invention is effected by administering to a subject in need thereof a therapeutically effective amount of a compound of the present invention, as described hereinabove, either per se, or, more preferably, as a part of a pharmaceutical composition, mixed with, for example, a pharmaceutically acceptable carrier, as is detailed hereinunder.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “administering” as used herein refers to a method for bringing a compound of the present invention and a target EGFR together in such a manner that the compound can affect the catalytic activity of the EGFR-TK either directly; i.e., by interacting with the kinase itself or indirectly; i.e., by interacting with another molecule on which the catalytic activity of the kinase is dependent. As used herein, administration can be accomplished either in vitro, i.e. in a test tube, or in vivo, i.e., in cells or tissues of a living organism.

Herein, the term “treating” includes abrogating, substantially inhibiting, slowing or reversing the progression of a disease or disorder, substantially ameliorating clinical symptoms of a disease or disorder or substantially preventing the appearance of clinical symptoms of a disease or disorder.

Herein, the term “preventing” refers to a method for barring an organism from acquiring a disorder or disease in the first place.

The term “therapeutically effective amount” refers to that amount of the compound being administered which will relieve to some extent one or more of the symptoms of the disease or disorder being treated.

For any compound used in this method of the invention, a therapeutically effective amount, also referred to herein as a therapeutically effective dose, can be estimated initially from cell culture assays. For example, a dose can be formulated in animal models to achieve a circulating concentration range that includes the IC₅₀ or the IC₁₀₀ as determined in cell culture. Such information can be used to more accurately determine useful doses in humans. Initial dosages can also be estimated from in vivo data. Using these initial guidelines one having ordinary skill in the art could determine an effective dosage in humans.

Moreover, toxicity and therapeutic efficacy of the radiolabeled compounds described herein can be determined by standard pharmaceutical procedures in cell cultures or experimental animals, e.g., by determining the LD₅₀ and the ED₅₀. The dose ratio between toxic and therapeutic effect is the therapeutic index and can be expressed as the ratio between LD₅₀ and ED₅₀. Compounds which exhibit high therapeutic indices are preferred. The data obtained from these cell cultures assays and animal studies can be used in formulating a dosage range that is not toxic for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, chapter 1, page 1).

Dosage amount and interval may be adjusted individually to provide plasma levels of the active compound which are sufficient to maintain therapeutic effect. Usual patient dosages for oral administration range from about 50-2000 mg/kg/day, commonly from about 100-1000 mg/kg/day, preferably from about 150-700 mg/kg/day and most preferably from about 250-500 mg/kg/day. Preferably, therapeutically effective serum levels will be achieved by administering multiple doses each day. In cases of local administration or selective uptake, the effective local concentration of the drug may not be related to plasma concentration. One having skill in the art will be able to optimize therapeutically effective local dosages without undue experimentation.

As used herein, “EGFR-TK related disease or disorder” refers to a disease or disorder characterized by inappropriate EGFR-TK activity or over-activity of the EGFR-TK. Inappropriate activity refers to either; (i) EGFR-TK expression in cells which normally do not express EGFR-TKs; (ii) increased EGFR-TK expression leading to unwanted cell proliferation, differentiation and/or growth; or, (iii) decreased EGFR-TK expression leading to unwanted reductions in cell proliferation, differentiation and/or growth. Over-activity of EGFR-TKs refers to either amplification of the gene encoding a particular EGFR-TK or production of a level of EGFR-TK activity which can correlate with a cell proliferation, differentiation and/or growth disorder (that is, as the level of the EGFR-TK increases, the severity of one or more of the symptoms of the cellular disorder increases). Over activity can also be the result of ligand independent or constitutive activation as a result of mutations such as deletions of a fragment of a EGFR-TK responsible for ligand binding.

Preferred diseases or disorders that the compounds described herein may be useful in preventing, treating and studying are cell proliferative disorders, such as, but not limited to, papilloma, blastoglioma, Kaposi's sarcoma, melanoma, lung cancer, ovarian cancer, prostate cancer, squamous cell carcinoma, astrocytoma, head cancer, neck cancer, bladder cancer, breast cancer, lung cancer, colorectal cancer, thyroid cancer, pancreatic cancer, gastric cancer, hepatocellular carcinoma, leukemia, lymphoma, Hodgkin's disease and Burkitt's disease.

Hence, further according to the present invention there is provided a method of inhibiting cell proliferation by subjecting the cells to any of the compounds described hereinabove. In a preferred embodiment of the invention the cells are of an organism (e.g., a human), whereas subjecting the cells to the compound is effected in vivo. Alternatively, subjecting the cells to the compound is effected in vitro.

Radiolabeled Compounds:

As is discussed hereinabove, and is further described hereinbelow, irreversible EGFR-TK inhibitors are particularly useful in diagnostic applications such as radioimaging. The novel compounds of the present invention were therefore designed so as to allow radiolabeling thereof at various positions by various radioisotopes. As is exemplified in the Examples section that follows, representative examples of radiolabeled compounds according to the present invention were successfully prepared.

Hence, according to another aspect of the present invention there is provided a radiolabeled compound having the general Formula III: 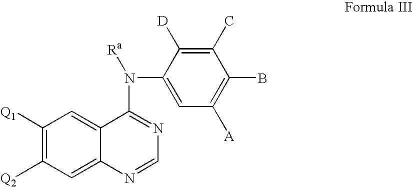
wherein:
Q1 is X—W(═Y)-Z and Q2 is selected from the group consisting of hydrogen, halogen, alkoxy, hydroxy, thiohydroxy, thioalkoxy, alkylamino and amino, or
Q1 is selected from the group consisting of hydrogen, halogen, alkoxy, hydroxy, thiohydroxy, thioalkoxy, alkylamino and amino and Q2 is X—W(═Y)-Z;
X is selected from the group consisting of —NR¹—, —O—, —NH—NR¹—, —O—NR¹—, NH—CHR¹—, —CHR¹—NH—, —CHR¹—O—, —O—CHR¹—, —CHR¹—CH₂— and —CHR¹—S— or absent;
W is carbon;
Y is selected from the group consisting of oxygen and sulfur;
Z is —CR²R³R⁴;
R^{a} is selected from the group consisting of hydrogen or alkyl having 1-8 carbon atoms;
A, B, C and D are each independently selected from the group consisting of hydrogen, a first non-radioactive derivatizing group and a first radioactive derivatizing group selected from a radioactive bromine, a radioactive iodine and a radioactive fluorine;
R¹ is selected from the group consisting of hydrogen, and substituted or non-substituted alkyl having 1-6 carbon atoms;
R² is a leaving group; and
R³ and R⁴ are each independently selected from the group consisting of hydrogen, a second non-radioactive derivatizing group and a second radioactive derivatizing group containing a radioactive fluorine, a radioactive bromine, a radioactive iodine and/or a radioactive carbon;
provided that the compound comprises at least one radioactive atom.

As used herein, the phrase “radiolabeled compound” or “radioactive atom” (type specified or not) refer to a compound that comprises one or more radioactive atoms or to a radioactive atom with a specific radioactivity above that of background level for that atom. It is well known, in this respect, that naturally occurring elements are present in the form of varying isotopes, some of which are radioactive isotopes. The radioactivity of the naturally occurring elements is a result of the natural distribution of these isotopes, and is commonly referred to as a background radioactive level. However, there are known methods of enriching a certain element with isotopes that are radioactive. The result of such enrichment is a population of atoms characterized by higher radioactivity than a natural population of that atom, and thus the specific radioactivity thereof is above the background level.

Thus, the radiolabeled compounds of the present invention have a specific radioactivity that is higher than the corresponding non-labeled compounds, and can therefore be used as agents for radioimaging and radiotherapy.

Furthermore, the term “non-radioactive”, as used herein with respect to an atom or a derivatizing group, refers to an atom or a derivatizing group, as this phrase is defined hereinabove, that does not comprise a radioactive atom and thus the specific radioactivity thereof is of a background level.

The term “radioactive”, as used herein with respect to an atom or a derivatizing group, refers to an atom or a derivatizing group that comprises a radioactive atom and therefore the specific radioactivity thereof is above the background level.

Preferred radiolabeled compounds according to the present invention include the preferred compounds described hereinabove, radiolabeled by one or more of a radioactive carbon, a radioactive fluorine, a radioactive bromine and a radioactive iodine.

The radioactive carbon is preferably carbon-11. The radioactive fluorine is preferably fluorine-18. The radioactive bromine can be bromine-76 or bromine-77. The radioactive iodine can be iodine-123, iodine-124 and iodine-131. According to a preferred embodiment of the invention, at least one of A, B, C and D is a radioactive fluorine, and the radioactive fluorine is fluorine-18. Preferably, D is fluorine-18. Thus, preferred fluorine-18 labeled compounds according to the present invention include fluorine-18 labeled Compounds 5 and 6.

According to another preferred embodiment of the present invention, the radioactive atom is a radioactive bromine such as bromine-76 and bromine-77. Preferably, A is the radioactive bromine. Thus, preferred radioactive bromine labeled compounds according to the present invention include bromine-76 and bromine-77 labeled Compounds 1 and 2. A bromine-76 labeled compound of the invention can be used for PET radioimaging, while a bromine-77 labeled compound of the invention can be used for radiotherapy.

According to yet another preferred embodiment of the present invention, the radioactive atom is a radioactive iodine such as iodine-123, iodine-124 or iodine-131. Preferably, A is the radioactive iodine. Thus, preferred radioactive iodine labeled compounds according to the present invention include iodine-123, iodine-124 and iodine-131 labeled Compounds 3 and 4.

An iodine-123 labeled compound of the invention can be used for SPECT radioimaging, an iodine-124 labeled compound of the invention can be used for both PET radioimaging and/or radiotherapy and an iodine-131 labeled compound of the invention can be used for radiotherapy.

The presently most preferred radiolabeled compounds according to the present invention are the iodine-124 labeled Compounds 3 and 4. The iodine-124 radioisotope is becoming increasingly significant in PET diagnostic use. It decays (t_{1/2}=4.2 days) simultaneously by positron emission (25.6%) and by electron capture (74.4%). Due to its quantity of short-range Auger electrons (9.2/decay) it has also been discussed as a potential therapeutic nuclide.

The substantially longer half-life of this isotope, as compared with the other optional radioisotopes considered, enables a prolonged follow up after injection of the radiolabeled compound. Following autophosphorylation of the receptor, it is degraded with a half-life of 20 hours, thus allowing sufficient receptor-inhibitor binding time for imaging.

In addition to the above, the radiolabeled compounds of the present invention can include a radioactive atom at the carboxylic side chain (represented by X—W(═Y)-Z in Formula III above), such that one or both of R³ and R⁴ are a radioactive derivatizing group, (defined herein as a second radioactive derivatizing group), which includes any of the radioactive atoms described hereinabove. The second derivatizing group can be, for example, a radioactive fluorine (e.g., fluorine-18) labeled, a radioactive bromine (e.g., bromine-76 or bromine-77) labeled, or a radioactive iodine (e.g., iodine-123, iodine-124 or iodine-131) labeled haloalkyl, cycloalkyl (substituted thereby), or aryl (substituted thereby). Alternatively, the second derivatizing group can be, for example, a radioactive carbon (e.g., carbon-11) labeled alkyl, haloalkyl, cycloalkyl, aryl, heteroaryl, carboxy, carbonyl and carbamyl.

Radiosyntheses:

According to another aspect of the present invention, there are provided methods for the syntheses of the radiolabeled compounds of the invention.

The radiolabeling of the compounds can be performed using four alternative strategies as follows:

The first strategy involves the incorporation of fluorine-18 atom within the aniline ring and requires that the radiolabeling be the first step of a multi-step radiosynthesis, which typically includes a total of four- to eight-step radiosynthesis, as is further exemplified in the Examples section that follows.

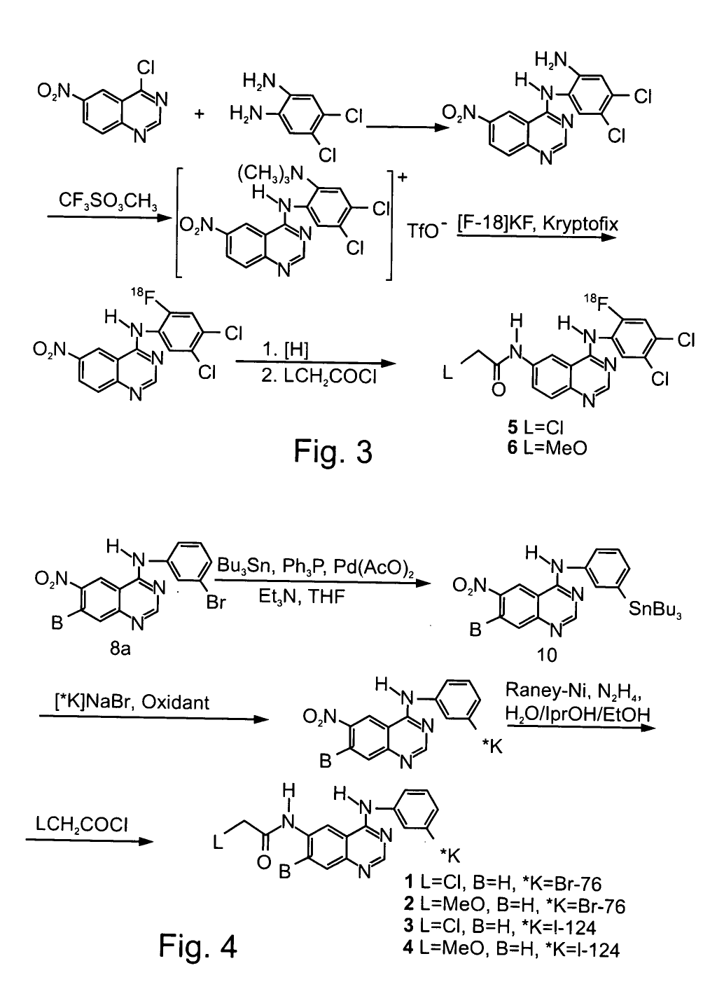

The second strategy also involves the incorporation of fluorine-18 atom within the aniline ring. However, in this newly developed strategy, which is presented in FIG. 3, the radiolabeling is performed two steps prior to the final step of the synthesis, thus being a more advantageous three-steps radiosynthesis.

The third strategy for radiolabeling according to the present invention involves the incorporation of a carbon-11 atom within the α-substituted carboxylic residue which is performed at the final step of the synthesis, thus being an advantageous one-step radiosynthesis.

The fourth strategy involves the incorporation of radioactive bromine or radioactive iodine within the anilino ring of the 4-(phenylamino)quinazoline, prior to the final step of the synthesis, resulting in an advantageous two-step radiosynthesis. General and detailed radiosynthesis procedures, based on the strategies above, are described in the Examples section that follows.

As is demonstrated in the Examples section that follows, using these strategies, representative examples of fluorine-18 labeled and iodine-124 labeled compounds according to the present invention have been successfully radiosynthesized.

Radioimaging and Radiotherapy:

The radiolabeled compounds herein described can be used as radioimaging and radiotherapy agents. Carbon-11 labeled, fluorine-18 labeled, bromine-76 labeled and iodine-124 labeled compounds of the invention can be used as biomarkers for PET radioimaging, whereas iodine-123 labeled compounds of the invention can be used as biomarkers for SPECT radioimaging. Bromine-77 labeled, iodine-124 and iodine-131 labeled compounds of the invention can be used as radiopharmaceuticals for radiotherapy. Thus, the radiolabeled compounds of the invention can be used to effect a method of monitoring the level of epidermal growth factor receptor within a body of a patient by administering to the patient any of the carbon-11, fluorine-18, bromine-76, iodine-123 or iodine-124 radiolabeled compounds described herein and employing a nuclear imaging technique, such as positron emission tomography or single photon emission computed tomography, for monitoring a distribution of the compound within the body or within a portion thereof.

Nuclear imaging dosing depends on the affinity of the compound to its receptor, the isotope employed and the specific activity of labeling. Persons ordinarily skilled in the art can easily determine optimum nuclear imaging dosages and dosing methodology.

The bromine-77, iodine-124 and iodine-131 radiolabeled compounds herein described can be used to effect a method of radiotherapy by administering to a patient a therapeutically effective amount, as is defined hereinabove, of a radiolabeled compound as described herein, either per se, or, preferably in a pharmaceutical composition, mixed with, for example, a pharmaceutically acceptable carrier.

Pharmaceutical Compositions:

Any of the compounds described herein, non-labeled and radiolabeled, can be formulated into a pharmaceutical composition which can be used for therapy of a disease or disorder (e.g., cancer therapy), radiotherapy of a disease or disorder or for imaging. Such a composition includes as an active ingredient any of the compounds described herein and a pharmaceutically acceptable carrier.

As used herein a “pharmaceutical composition” refers to a preparation of one or more of the compounds described herein, with other chemical components such as pharmaceutically suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water. Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition.

Routes of administration: Suitable routes of administration may, for example, include oral, rectal, transmucosal, transdermal, intestinal or parenteral delivery, including intramuscular, subcutaneous and intramedullary injections as well as intrathecal, direct intraventricular, intravenous, intraperitoneal, intranasal, or intraocular injections.

Composition/formulation: Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer with or without organic solvents such as propylene glycol, polyethylene glycol. For transmucosal administration, penetrants are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the compounds for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile, pyrogen-free water, before use.

The compounds of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

The pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a compound of the invention formulated in a compatible pharmaceutical carrier may also be prepared, placed in an appropriate container, and labeled for treatment of an indicated condition. Suitable conditions indicated on the label may include treatment of cell proliferation disease or disorder such as certain cancers associated with EGFR-TK activity, and radioimaging.

Hence, according to a preferred embodiment of the present invention, the pharmaceutical composition described hereinabove is packaged in a packaging material and identified in print, in or on the packaging material for use in the treatment of an EGFR-TK related disease or disorder, as is described hereinabove.

Additional objects, advantages, and novel features of the present invention will become apparent to one ordinarily skilled in the art upon examination of the following examples, which are not intended to be limiting. Additionally, each of the various embodiments and aspects of the present invention as defined hereinabove and as claimed in the claims section below finds experimental support in the following examples.

### EXAMPLES

Reference is now made to the following examples, which together with the above descriptions, illustrate the invention in a non-limiting fashion.

### Materials, Syntheses and Experimental Methods

Chemical Syntheses:

All chemicals were purchased from Sigma-Aldrich, Fisher Scientific, Merck or J. T. Baker. Chemicals were used as supplied, excluding DMSO, which was stored over activated molecular sieves for at least one day prior to use, THF, which was refluxed over sodium and benzophenone, and freshly distilled prior to use, and vinyl magnesium which was freshly prepared by reacting vinyl bromide and magnesium turnings, according to well-known procedures, prior to use.

Mass spectrometry was performed in EI mode on a Thermo Quest-Finnigan Trace MS-mass spectrometer at the Hadassah-Hebrew University Mass Spectroscopy facility.

¹H-NMR spectra were obtained on a Bruker AMX 300 MHz instrument.

Elemental analysis was performed at the Hebrew University Microanalysis Laboratory.

HPLC analyses of the labeled and unlabeled compounds were performed on a reversed-phase system using Waters γ-Bondapack C18 analytical column (10 μm, 300×3.9 mm) with mobile phase systems, composed of CH₃CN/acetate buffer or 47% CH₃CN/53% 0.1 M ammonium formate buffer.

6-Nitroquinazolone was prepared according to a published procedure (Elderfield et al., 1947).

Microwave heating was performed in a conventional oven (BR 740XL, Brother) operating at 500 W (full power).

### Synthesis of N-[4-(phenylamino)quinazoline-6-yl]amides Substituted by a Leaving Group at the α Position—General Procedure

Aniline or derivatized aniline (1 equivalent) is reacted with 4-chloro-6-nitroquinazoline (3.5 equivalents), in a polar solvent such as iso-propylalcohol. The product, 6-nitro-4-(phenylamino)quinazoline, is obtained after filtration. A solution of 6-nitro-4-(phenylamino)quinazoline in ethanol/water and a polar solvent such as iso-propylalcohol is thereafter reacted at reflux temperature with hydrazine hydrate and Raney®Nickel. The reaction mixture is filtered, evaporated and purified by silica gel chromatography, to give 6-amino-4-(phenylamino)quinazoline. 6-Amino-4-(phenylamino)quinazoline is then reacted with a reactive carboxylic derivative substituted at the α position by a leaving group, and optionally by a derivatazing group, at 0° C. in THF, in the presence of a chemically reactive base such as tertiary amine, to give the final product.

Optionally, N-[4-(phenylamino)quinazoline-6-yl]amides substituted by a leaving group and further substituted at the quinozaline ring by a morpholino or piperazino group can be synthesized according to the following representative general procedure:

Aniline or derivatized aniline (1 equivalent) is reacted with 4-chloro-7-fluoro-6-nitroquinazoline (3.5 equivalents), in a polar solvent such as iso-propylalcohol. The product, 6-nitro-7-fluoro-4-(phenylamino)quinazoline, is obtained after filtration. Sodium metal (5 equivalents) is added, under nitrogen atmosphere, to a solution of 3-(4-morpholinyl)-1-propanol (4 equivalents) in THF. The obtained suspension is stirred at 20° C. for two hours and is thereafter cannulated, under nitrogen atmosphere, into a solution of a 6-nitro-7-fluoro-4-(phenylamino)quinazoline. The reaction mixture is refluxed for 18 hours, the solvent is thereafter partially removed under reduced pressure and the residue is diluted with water and extracted with ethyl acetate. The combined organic extracts are dried, evaporated and purified on silica gel chromatography, to give 6-nitro-4-(phenylamino)-7-[3-(4-morpholinyl)propoxy]-quinazoline. The 6-nitro-4-(phenylamino)-7-[3-(4-morpholinyl)propoxy]-quinazoline is thereafter reacted with hydrazine hydrate and Raney®Nickel, as described hereinabove, to produce 6-amino-4-(phenylamino)-7-[3-(4-morpholinyl)propoxy]-quinazoline, which is further reacted with a reactive carboxylic derivative substituted by a leaving group in THF, at 0° C., in the presence of a base, to yield the final 7-morpholino-substituted product.

Thus, according to the general pathway described above, 4-(phenylamino)quinazolines substituted by the following carboxylic side-chain groups substituted at the α position by a leaving group, and optionally by a derivatizing group, are synthesizable:

Amine-linked side-chains: 4-(phenylamino)quinazoline substituted at position 6 or 7 by a nitro group is reduced to the corresponding amine, which is then acylated by a carboxylic acid substituted at the a position by a leaving group in the presence of a coupling agent, such as EI or AC, or by the acid chloride.

Oxygen-linked side-chains: 4-(phenylamino)quinazoline substituted at position 6 or 7 by a methoxy group is cleaved to produce the corresponding hydroxyl compound, which is then acylated either by a carboxylic acid substituted at the α position by a leaving group in the presence of a coupling agent such as EDAC, or by the acid chloride.

Carbon-linked side-chains: 4-(phenylamino)quinazoline substituted at position 6 or 7 by iodine is converted to the corresponding arylzinc compound which is coupled with a carboxylic group substituted at the α position by a leaving group that comprises an activated halide.

Hydrazino-linked side-chains: 4-(phenylamino)quinazoline substituted at position 6 or 7 by a nitro group is reduced to the corresponding amine, which is diazotized and then reduced to the hydrazine compound. The distal nitrogen of the hydrazine is then acylated, using methods well known to one skilled in the art, by an appropriate carboxylic derivative substituted at the α position by a leaving group.

Hydroxylamino-O-linked side-chains. 4-(phenylamino)quinazoline substituted at position 6 or 7 by a nitro group is reduced under appropriate mildly reducing conditions to the hydroxylamine compound which is then acylated, using methods well-known to one skilled in the art, by an appropriate carboxylic derivative substituted at the α position by a leaving group.

Methyleneamino-N-linked side-chains. 4-(phenylamino) quinazoline substituted at position 6 or 7 by a nitro group is reduced to the corresponding amine which is diazotized and then converted to nitrile, preferably in the presence of copper or nickel salt catalysis. The nitrile compound is then reduced to a methylamine compound which is acylated, using methods well known to one skilled in the art, by an appropriate carboxylic derivative substituted at the α position by a leaving group.

Methyleneoxy-O-linked side-chains: 4-(phenylamino)quinazoline substituted at position 6 or 7 by a hydroxymethyl is produced using methods obvious to one skilled in the art. For example, 4-(phenylamino)quinazoline substituted at position 6 or 7 by a nitro group is reduced to the corresponding amine which is diazotized, converted to the nitrile as described above, partially reduced to an imine, hydrolyzed and reduced to the corresponding hydroxymethyl. The hydroxyl group is then acylated, using methods well known to one skilled in the art, by an appropriate carboxylic derivative substituted at the a position by a leaving group.

Ethano-linked side-chains: 4-(phenylamino)quinazoline substituted at position 6 or 7 by iodine is converted, via an organozincate, to the corresponding cuprate. The cuprate is reacted with an appropriate divinylketone substituted at the α position by a leaving group, which is then subjected to unmasking of the unsaturated functionality.

Aminomethyl-C-linked side-chains: 4-(phenylamino)quinazoline substituted at position 6 or 7 by a nitro group is reduced to the corresponding amine which is alkylated by a derivative of an appropriate saturated ketone substituted at the a position by a leaving group.

Hydroxymethyl-C-linked side-chains. 4-(phenylamino)quinazoline substituted at position 6 or 7 by a methoxy group is cleaved to the corresponding hydroxyl compound which is alkylated by an appropriate saturated ketone substituted at the α position by a leaving group.

Thiomethyl-C-linked side-chains: 4-(phenylamino)quinazoline substituted at position 6 or 7 by halide is converted to the corresponding mercapto compound which is then alkylated by an appropriate saturated ketone substituted at the α position by a leaving group.

Based on the general procedure described above, representative examples of 6-nitro-4-(phenylamino)-quinazolines and their corresponding 6-amino-4-(phenylamino)-quinazolines were synthesized as follows:

### Synthesis of 4-chloro-6-nitroquinazoline

6-Nitroquinazolone (2 grams, 0.01 mmol) and SOCl₂ (20 ml) were placed in a two-necked flask and DMF (100 μl) was added. The mixture was refluxed for 1 hour, and then additional quantities of SOCl₂ (10 ml) and DMF (50 μl) were added. After a 3 hours reflux the thionyl chloride was distilled out, and the purity of the product, 4-chloro-6-nitroquinazoline was determined using a reversed-phase C18 analytical HPLC column (96-98% purity). The compound was kept at 0° C., and used without any further purification for the next step.

Mp=130° C.;

¹H-NMR (DMSO-_{d6}): δ=8.78 (1H, d, J=2 Hz), 8.555 (1H, dd, J1=6.7 Hz, J2=2 Hz), 8.432 (1H, s), 7.883 (1H, d, J=6.7 Hz);

HPLC conditions: C18 analytical column, 40% acetate buffer pH=3.8/60% acetonitrile, flow=1 ml/minute; Rₜ=4.95 minutes.

### Synthesis of 6-nitro-4-[(3-iodophenyl)amino]-quinazoline

4-chloro-6-nitroquinazoline, prepared as described hereinabove (4 grams, 23 mmol) and 3-iodoaniline (12.57 grams, 57 mmol) were dissolved and stirred in i-PrOH (40 ml) at 25° C. for 10 minutes, yielding a bright-yellow precipitate. The mixture was then refluxed, stirred for an additional 3 hours, and cooled. The solid was filtered, rinsed with i-PrOH (12 ml), and dried in a vacuum oven at 80° C. to yield the product (5.99 grams, 78%).

MS (m/z): 393.2 (MH)⁺;

¹H-NMR (DMSO-d₆): δ=10.56 (1H, s), 9.664 (1H, d, J=2.4 Hz), 8.784 (1H, s), 8.578 (1H, dd, J1=11.4 Hz, J2=2.1 Hz), 8.270 (1H, bs), 7.955 (2H, m), 7.543 (1H, d, J=8.1 Hz), 7.228 (1H, t, J=7.8 Hz);

HPLC conditions: C18 analytical column, 45% acetate buffer pH=3.8/55% acetonitrile, flow=1 ml/minute; Rₜ=17.8 minutes.

### Synthesis of 6-amino-4-[(3-iodophenyl)amino]-quinazoline

6-Amino-4-[(3-iodophenyl)amino]-quinazoline, prepared as described hereinabove, (620 mg, 1.58 mmol) was placed in a flask, and a solution of H₂O:EtOH:IPA, 5%:45%:50% (107 ml) was added. The mixture was heated to 95° C., and an additional 50 ml of solvent was added until complete dissolution. The mixture was cooled to 65° C., and RaNi (½ Pasteur pipette) and hydrazine hydrate (153 μl, 3.16 mmol) were added successively until a green solution was obtained. The reaction was heated to 80-85° C., and more RaNi (½ Pasteur pipette) and hydrazine hydrate (38 μl, 0.8 mmol) were added. Reflux was maintained for 15-20 minutes. The solution was cooled, and filtered through a layer of celite (prepared as slurry in EtOH). The mixture was evaporated to yield the product (180 mg, 31.4%).

MS (m/z): 363.0 (MH)⁺;

¹H-NMR (DMSO-_{d6}): δ=9.365 (1H, s), 8.347 (1H, s), 8.323 (1H, t, J=2.4 Hz), 7.918 (1H, dd, J1=10 Hz, J2=2.4 Hz), 7.524 (1H, d, J=11.6 Hz), 7.388 (1H, d, J=7.2 Hz), 7.318 (1H, d, J=2.8 Hz) 7.235 (1H, dd, J1=11.6 Hz, J2=2.8 Hz), 7.134 (1H, t, J=10.4 Hz) 5.595 (2H, bs);

HPLC conditions: C18 analytical column, 55% acetate buffer pH=3.8/45% acetonitrile, flow=1 ml/minute; Rₜ=8.3 minutes.

### Synthesis of 6-nitro-4-[(3-bromophenyl)amino]-quinazoline

This compound was prepared as described hereinabove for the corresponding 3-iodophenylamino quinazoline, by reacting 4-chloro-6-nitroquinazoline and 3-bromo aniline.

m.p.=267-270° C.;

MS (m/z): 345 (MH)⁺;

HPLC conditions: C18 column, 55% acetate buffer pH=3.8/45% acetonitrile, flow=1 ml/minute; Rₜ=7.54 minutes.

### Synthesis of 6-amino-4-[(3-bromophenyl) amino]-quinazoline

This compound was prepared from 6-nitro-4-[(3-bromophenyl)amino]-quinazoline (590 mg, 1.7 mmol) as described above for the corresponding iodoquinazoline (332 mg, 62%).

m.p.=204° C.;

MS (m/z): 315 (MH)⁺;

HPLC conditions: C18 column, 45% acetate buffer pH=3.8/55% acetonitrile, flow=1 ml/minute; Rₜ=6.41 minutes.

### Synthesis of 6-nitro-4-[(4,5-dichloro-2-fluoro-phenyl)amino]quinazoline

3,4-Dichloro-6-fluoroaniline (1 equivalent, prepared as described in U.S. Pat. No. 6,126,917) was reacted with 4-chloro-6-nitroquinazoline (3.5 equivalents, prepared as described hereinabove), in iso-propylalcohol. After filtration, 6-nitro-4-[(3,4-dichloro-6-fluorophenyl)amino]-quinazoline was obtained in 60% yield.

m.p.=270-271° C.;

MS (m/z): 353.2, 355.2 (M⁺);

¹H-NMR: δ=6.97 (d, 1H), 7.345 (d, 1H), 7.885 (d, 1H), 8.405 (d, 1H), 8.554 (dd, 1H), 8.8 (d, 1H).

HPLC conditions: C-18 column, 55% acetate buffer, PH=3.8/45% acetonitrile, flow=1 ml/minute; r.t.=7.15 minutes.

### Synthesis of 6-amino-4-[(4,5-dichloro-2-fluoro-phenyl)amino]quinazoline

A solution of 6-nitro-4-[(3,4-dichloro-6-fluorophenyl)amino]-quinazoline (709 mg, 2.076 mmol) in 140 ml of 1:9:10 water:ethanol:iso-propylalcohol was heated to reflux temperature (95° C.). Additional 60 ml of the solvents mixture was added until complete dissolution. The reaction mixture was then cooled to 65° C., and 200 μl hydrazine hydrate (4.12 mmol) and 0.5 ml Raney®Nickel (in water) were added subsequently thereto. The resulting mixture was heated up to 80-85° C., additional 0.5 ml Raney®Nickel and 50 μl of hydrazine hydrate (1.03 mmol) were added, and gentle reflux was maintained for about 15-20 minutes. Filtration and evaporation gave 6-amino-4-[(3,4-dichloro-6-fluorophenyl)amino]-quinazoline in 83% yield.

m.p.=265° C.;

MS (m/z): 323.4, 325.4 (M⁺);

Anal. calcd.: C, 52.9; H, 2.78; N, 17.33. Found: C, 52.19; H, 2.99; N, 17.14;

HPLC analysis: C-18 column, 55% acetate buffer, PH=3.8/45% acetonitrile, flow=1 ml/minute; r.t=6.6 minutes.

The compounds above were used for the syntheses of representative examples of [4-(phenylamino)quinazoline-6-yl]amides substituted by a leaving group, as follows:

### Synthesis of N-{4-[(3-bromophenyl)amino]-quinazolin-6-yl}-2-chloroacetamide (Compound 1)

To a stirred solution of 6-amino-4-[(3-bromophenyl)amino]quinazoline (120 mg, 0.38 mmol, prepared as described hereinabove) in dry THF, at 0° C. and under nitrogen atmosphere, N,N-diisopropylethylamine (193 μl, 1.1 mmol) was added, followed by addition of chloroacetyl chloride (88 μl, 1.1 mmol). The mixture was stirred at 0° C. for 0.5 hour and was then poured into saturated NaHCO₃ and extracted with EtOAc. The organic solution was dried (Na₂SO₄) and evaporated. The residue was chromatographed on silica gel. Elution with 3% MeOH/97% CH₂CL₂ gave 121 mg (81% yield) of N-{4-[(3-Bromophenyl)amino]-quinazolin-6-yl}-2-chloro-acetamide.

m.p.>300° C.;

¹H-NMR[(CD₃)₂SO]: δ=10.6 (s, 1H), 9.97 (s, 1H), 8.71 (s, 1H), 8.6 (s, 1H), 8.15 (m, 1H), 7.8 (m, 2H), 7.31 (m, 3H), 4.34 (s, 2H);

MS m/e: 393 (100%, MH₂⁺), 391 (99%, MH⁺);

Anal. (C₁₆H₁₂BrClN₄O): calcd.: C, 49.07; H, 3.09; N, 14.31. Found: C, 48.94; H, 3.15; N, 13.66.

### Synthesis of N-{4-[(3-bromophenyl)amino]-quinazolin-6-yl}-2-methoxyacetamide (Compound 2)

Methoxyacetyl chloride (37 mg, 0.34 mmol) was added to a stirred solution of 6-amino-4-[(3-bromophenyl)amino]quinazoline (63 mg, 0.2 mmol, prepared as described hereinabove) and triethylamine (34 mg, 0.34 mmol) in THF (20 ml), at 0° C. The mixture was stirred at 0° C. for 0.5 hour and was then poured into saturated NaHCO₃ and extracted with EtOAc. The organic solution was dried (Na₂SO₄) and evaporated. The residue was chromatographed on silica gel. Elution with 3% MeOH/97% CH₂Cl₂ gave 53 mg (69% yield) of N-{4-[(3-bromophenyl)amino]-quinazolin-6-yl}-2-methoxyacetamide.

m.p.=190-191° C.;

¹H-NMR[(CD₃)₂SO]: δ=10.1 (s, 1H), 9.9 (s, 1H), 8.72 (d, J=3.6 Hz, 1H), 8.6 (s, 1H), 8.2 (t, J=3.6 Hz, 1H), 8.01 (dd, J₁=16 Hz, J₂=3.6 Hz, 1H), 7.87 (dt, J₁=13 Hz, J₂=3.4, 1H), 7.82 (d, J=16 Hz, 1H), 7.3 (m, 2H), 4.1 (s, 2H), 3.4 (s, 3H);

MS m/e: 387 (100%, MH⁺), 389 (99%, MH⁺), 388 (19%, MH⁺), 390 (18%, MH⁺) 391 (3%, MH);

Anal. (C₁₇H₁₅BrN₄O₂): calcd.: C, 52.68; H, 3.87; N, 14.46. Found: C, 52.47; H, 4.19; N, 14.06.

### Synthesis of N-{4-[(3-iodophenyl)amino]-quinazolin-6-yl}-2-chloroacetamide (Compound 3)

To a stirred solution of 6-amino-4-[(3-iodophenyl)amino]quinazoline (138 mg, 0.38 mmol, prepared as described hereinabove) in dry THF, at 0° C. and under nitrogen atmosphere, N,N-diisopropylethylamine (166 μl, 0.95 mmol) was added, followed by addition of chloroacetyl chloride (76 μl, 0.94 mmol). The mixture was stirred at 0° C. for 0.5 hour and was then poured into saturated NaHCO₃ and extracted with EtOAc. The organic solution was dried (Na₂SO₄) and evaporated. The residue was chromatographed on silica gel. Elution with 3% MeOH/97% CH₂CL₂ gave 90 mg (54% yield) of N-{4-[(3-iodophenyl)amino]-quinazolin-6-yl}-2-chloroacetamide.

m.p.>300° C.;

¹H-NMR[(CD₃)₂SO]: δ=10.6 (s, 1H), 9.97 (s, 1H), 8.71 (s, 1H), 8.6 (s, 1H), 8.25 (m, 1H), 7.8 (m, 2H), 7.41 (d, J=7.8 Hz, 1H), 7.17 (m, 2H), 4.34 (s, 2H);

MS m/e: 439 (100%, MH⁺);

Anal. (C₁₆H₁₂ClN₄O): calcd.: C, 43.81; H, 2.76; N, 12.77. Found: C, 43.54; H, 3.17; N, 12.21.

### Synthesis of N-{4-[(3-iodophenyl)amino]-quinazolin-6-yl}-2-methoxyacetamide (Compound 4)

4-Methoxyacetyl chloride (51 mg, 0.47 mmol) was added to a stirred solution of 6-amino-4-[(3-iodophenyl)amino]quinazoline (145 mg, 0.4 mmol, prepared as described hereinabove) and triethylamine (47 mg, 0.47 mmol) in THF (20 ml), at 0° C. The mixture was stirred at 0° C. for 0.5 hour and was then poured into saturated NaHCO₃ and extracted with EtOAc. The organic solution was dried (Na₂SO₄) and evaporated. The residue was chromatographed on silica gel. Elution with 3% MeOH/97% CH₂Cl₂ gave 102 mg (64% yield) of N-{4-[(3-iodophenyl)amino]-quinazolin-6-yl}-2-methoxyacetamide.

m.p.=159-163° C.;

¹H-NMR[(CD₃)₂SO]: δ=10.1 (s, 1H), 9.8 (s, 1H), 8.69 (d, J=3.7 Hz, 1H), 8.57 (s, 1H), 8.2 (t, J=3.3 Hz, 1H), 7.98 (dd, J₁=16.2 Hz, J₂=3.7 Hz, 1H), 7.9 (dm, J₁=14.7 Hz, 1H), 7.77 (d, J=16.2 Hz, 1H), 7.46 (dt, J=14.7 Hz, 1H), 7.18 (t, J=14.4 Hz, 1H), 4.1 (s, 2H), 3.4 (s, 3H);

MS: m/e=435 (100%, MH⁺);

Anal. (C₁₇H₁₅IN₄O₂): calcd.: C, 46.97; H, 3.45; N, 12.89. Found: C, 46.29; H, 3.65; N, 12.59.

### Synthesis of N-{4-[(4,5-Dichloro-2-fluoro-phenyl)amino]-quinazolin-6-yl}-2-chloroacetamide (Compound 5)

To a stirred solution of 6-amino-4-[(4,5-dichloro-2-fluoro-phenyl)amino]quinazoline (102 mg, 0.315 mmol, Ben David et al. 2003) in dry THF, at 0° C. and under nitrogen atmosphere, N,N-diisopropylethylamine (134 μl, 0.774 mmol) was added, followed by addition of chloroacetyl chloride (62 μl, 0.774 mmol). The mixture was stirred at 0° C. for 0.5 hour and was then poured into saturated NaHCO₃ and extracted with EtOAc. The organic solution was dried (Na₂SO₄) and evaporated. The residue was chromatographed on silica gel. Elution with 3% MeOH/97% CH₂CL₂ gave 93 mg (74% yield) of 2-chloro-N-{4-[(4,5-dichloro-2-fluoro-phenyl)amino]-quinazolin-6-yl}-2-chloroacetamide.

m.p.>300° C.;

¹H-NMR[(CD3)₂SO]: δ=10.6 (s, 1H), 10.1 (s, 1H), 8.7 (s, 1H), 8.47 (s, 1H), 7.8 (m, 4H), 4.3 (s, 2H);

MS: m/e=399 (100%, MH⁺);

Anal. (C₁₆H₁₀Cl₃FN₄O): calcd.: C, 48.03; H, 2.52; N, 14.03. Found: C, 47.51; H, 2.83; N, 13.43.

### Synthesis of N-{4-[(4,5-Dichloro-2-fluoro-phenyl)amino]-quinazolin-6-yl}-2-methoxyacetamide (Compound 6)

Methoxyacetyl chloride (42 mg, 0.39 mmol) was added to a stirred solution of 6-amino-4-[(4,5-dichloro-2-fluoro-phenyl)amino]quinazoline (62.4 mg, 0.193 mmol, Ben David et al. 2003) and triethylamine (39 mg, 0.386 mmol) in dry THF (20 ml), at 0° C. The mixture was stirred at 0° C. for 0.5 hour and was then poured into saturated NaHCO₃ and extracted with EtOAc. The organic solution was dried (Na₂SO₄) and evaporated. The residue was chromatographed on silica gel. Elution with 4% MeOH/96% CH₂Cl₂ gave 54 mg (71% yield) of N-{4-[(4,5-dichloro-2-fluoro-phenyl)amino]quinazolin-6-yl}-2-methoxyacetamide.

m.p.=204-206° C.;

¹H-NMR[(CD3)₂SO]: δ=10.1 (s, 1H), 9.9 (s, 1H), 8.7 (s, 1H), 8.5 (s, 1H), 7.9 (m, 4H), 4.1 (s, 2H), 3.4 (s, 3H);

MS: m/e=395 (100%, MH⁺), 397 (65%, MH⁺), 39 (19%, MH⁺);

Anal. (C₁₇H₁₃Cl₂FN₄O₂): calcd.: C, 51.61; H, 3.29; N, 14.53. Found: C, 51.74; H, 3.78; N, 13.93.

Radiosyntheses:

Generation of [F-18] Fluoride ion: ¹⁸F-Fluoride ion was produced by the ¹⁸O(p, n) ¹⁸F nuclear reaction on about 350 μl ¹⁸O-enriched water (97% isotopic purity, Rotem, Israel) as a target in the Hadassah-Hebrew University IBA 18/9 cyclotron (Belgium). Reactive organic ¹⁸F-fluoride ion was prepared by adding 10-50 μl irradiated target water to Kryptofix®2.2.2 (10 mg, 27 μl) and K₂CO₃ (1 mg) in water-acetonitrile. Azeotropic removal of water with acetonitrile was achieved by heating under a stream of nitrogen. The dried Kryptofix®2.2.2—potassium ¹⁸F-fluoride was then dissolved in 300 μl anhydrous DMSO for use in radiolabeling.

Generation of carbon-11 CO₂: [carbon-11]-CO₂ is produced by the ¹⁴N(p, α) ¹¹C nuclear reaction on a mixture of N₂/0.5% O₂ as a target.

Generation of iodine-124 sodium iodide: ¹²⁴I-NaI was purchased as a 0.02 M solution from Ritverc GmBH, Russia.

¹²⁴I-aminoquinazoline was prepared according to the general procedure of John et al. (1993).

HPLC separations were carried out using a Varian 9012Q pump, a Varian 9050 variable wavelength detector operating at 254 nm and a Bioscan Flow-Count radioactivity detector with a NaI crystal.

The carbon-11 labeled, fluorine-18 labeled, radioactive bromine labeled and radioactive iodine labeled compounds were purified on a reverse phase system using a C18-reverse phase-prep column and the following mobile phase system: 48% CH₃CN in 52% acetate buffer (pH=3.8), at 15 ml/minute flow rate. Eluent fractions (2.5 ml) were collected on a fraction collector (FC205, Gilson). Analysis of formulated radiotracers was performed on C18 column μ Bondapak analytical column, using 40% CH₃CN in 60% acetate buffer (pH=3.8) as elute, at a flow rate of 1.7 ml/min

Radiotracers formulation was performed as follows: The product was collected in a vial that contained 50 ml water and 1 ml NaOH (1 M). The solution was passed through a pre-washed (10 ml water) activated C18 cartridge, and washed with 10 ml sterile water. The product was eluted using 1 ml ethanol followed by 5 ml of saline.

### Synthesis of fluorine-18 Labeled [4-(phenylamino)quinazolin-6-yl]amides Substituted by a Leaving Group at the α Position—General Procedure I

The Kryptofix®2.2.2—potassium ¹⁸F-fluoride—DMSO solution described above is added to about 10 mg of a pre-selected dinitrobenzene in a screw-top test tube (8 ml, Corning). The tube is capped, shaken and heated in a microwave for 3.5 minutes. The tube is cooled in an ambient water bath, and the contents thereof are diluted with 10 ml of water and loaded onto an activated (ethanol) and equilibrated (water) C18 Sep-Pak (classic, short body, Waters). The cartridge is washed with water (10 ml) and the desired corresponding intermediate, fluorine-18 labeled fluoronitrobenzene, is eluted with ethanol (2 ml) into a small glass test tube. The reduction vessel is prepared by adding to a flat-bottomed glass vial (25 ml), sequentially, a few borosilicate glass beads, 100 μl 4:1 ethanol-water, 250 μl Raney®Nickel slurry, and 60 μl hydrazine monohydrate. After capping with a septum-equipped screw cap (vented with a large diameter needle) the vial is shaken and placed in a 40° C. heating block. The ethanolic fluorine-18 labeled fluoronitrobenzene solution is diluted with 0.5 ml water and added slowly to the reduction vessel. After 5 minutes, the vessel is cooled in an ambient water bath, and the vial content is filtered through a 0.45 μm filter (Puradisc, polypropylene, Whatman) into another flat-bottomed 25 ml vial. Eight ml of water and 10 ml of ether are then added to the filtered solution, and by capping and inverting several times to mix, the corresponding fluorine-18 labeled fluoroaniline reduction product is extracted into the ether layer. An 8 ml screw-top test tube is then charged with a solution of 4-5 mg of a 4-chloro-6-nitroquinazoline in 300 μl 2-propanol. The ethereal radiolabeled aniline solution is added to the tube by passing it through MgSO₄ (2 grams) and a new 0.45 μm filter. The ether is removed under a stream of helium, while warming the tube in an ambient water bath. Concentrated HCl (1 μl) is added thereafter and the capped tube is heated in a 110° C. oil bath for 15 minutes. After cooling the tube in ambient water, the acid is neutralized and the free base is liberated with the addition of 50 μl of 5M NaOH. Dichloromethane (0.3 ml) and hexane (0.3 ml) are added to the tube and the solution is filtered through a 0.2 μm filter (Acrodisc, nylon. Gelman). The fluorine-18 labeled 4-[(fluorophenyl)amino]-6-nitroquinazoline is purified by silica SEP-PAK and reduced to obtain the amine derivative thereof, which is further reacted with a reactive carboxylic derivative as described hereinabove.

Following are detailed syntheses of representative examples of a fluorine-18 labeled [4-(phenylamino)quinazolin-6-yl]amides substituted by a leaving group at the α position, prepared according to the general procedure I described hereinabove.

### Synthesis of fluorine-18 Labeled of N-{4-[(4,5-Dichloro-2-fluoro-phenyl)amino]-quinazolin-6-yl}-2-chloroacetamide (Fluorine-18 Labeled Compound 5)

Fluorine-18 labeled 4-[(3,4-dichloro-6-fluorophenyl)amino]-6-nitro quinazoline was obtained by the radiosynthesis procedure described hereinabove, using 10 mg of 1,2-dichloro-4,5-dinitrobenzene in the reaction with the ¹⁸F-fluoride ion ([¹⁸F]KF, 200 μl DMSO/200 μl CH₃CN, 20 minutes, 120° C., kryptofix) to provide 1,2-dichloro-4-¹⁸F-fluoro-5-nitrobenzene (80% yield). Following purification on a C18 sep-pak column and elution with 2 ml EtOH, 1,2-dichloro-4-¹⁸F-fluoro-5-nitrobenzene was reduced to the corresponding aniline as described hereinabove, by means of Raney®Nickel and hydrazine hydrate, for 5 minutes at 60° C. After filtration, addition of water (4 ml), ether extraction and evaporation, the fluorine-18 labeled aniline was reacted with 4-chloro-6-nitroquinazoline, in isopropanol for 20 minutes, as described. The fluorine-18 labeled 4-[(3,4-dichloro-6-fluorophenyl)amino]-6-nitroquinazoline was then reduced to the corresponding aminoquinazoline as described, by means of Raney®Nickel and hydrazine hydrate, for 5 minutes at 60° C., and was further reacted with α-chloroacetyl chloride in THF and a catalytic amount of Et₃N as described, to yield the final fluorine-18 labeled product (5% decay corrected radiochemical yield after HPLC purification with acetate buffer/CH₃CN).

### Synthesis of fluorine-18 Labeled of N-{4-[(4,5-Dichloro-2-fluoro-phenyl)amino]-quinazolin-6-yl}-2-methoxyacetamide. (Fluorine-18 Labeled Compound 6)

Fluorine-18 labeled 4-[(3,4-dichloro-6-fluorophenyl)amino]-6-nitro quinazoline was obtained by the radiosynthesis procedure described hereinabove, using 10 mg of 1,2-dichloro-4,5-dinitrobenzene in the reaction with the ¹⁸F-fluoride ion ([¹⁸F]KF, 200 μl DMSO/200 μl CH₃CN, 20 minutes, 120° C., kryptofix) to provide 1,2-dichloro-4-¹⁸F-fluoro-5-nitrobenzene (80% yield). The 1,2-dichloro-4-¹⁸F-fluoro-5-nitrobenzene was purified as described hereinabove and was thereafter reduced to the corresponding aniline, as described hereinabove, purified and s reacted with 4-chloro-6-nitroquinazoline as described hereinabove. The fluorine-18 labeled 4-[(3,4-dichloro-6-fluorophenyl)amino]-6-nitroquinazoline was reduced to the corresponding aminoquinazoline as described and was further reacted with α-methoxyacetyl chloride in THF and a catalytic amount of Et₃N as described to yield the final fluorine-18 labeled product (5% decay corrected radiochemical yield after HPLC purification with acetate buffer/CH₃CN).

### Synthesis of fluorine-18 Labeled N-{4-[(3,4-dichloro-6-fluorophenyl)amino]-7-[3-(4-morpholinyl)propoxy]quinazoline-6-yl}2-chloro/2-methoxyacetamide (fluorine-18 Labeled Morpholino-Substituted Compounds 5 and 6)

Fluorine-18 labeled 4-[(3,4-dichloro-6-fluorophenyl)amino]-7-fluoro-6-nitroquinazoline is obtained by the radiosynthesis procedure described hereinabove, using 1,2-dichloro-4,5-dinitrobenzene in the reaction with the ¹⁸F-fluoride ion to provide 1,2-dichloro-4-¹⁸F-fluoro-5-nitrobenzene, which is reduced to the corresponding aniline. The obtained aniline is reacted with 4-chloro-7-fluoro-6-nitroquinazoline as described. The fluorine-18 labeled 4-[(3,4-dichloro-6-fluorophenyl)amino]-7-fluoro-6-nitroquinazoline is then reacted with the sodium salt of 3-(4-morpholinyl)-1-propanol as described hereinabove and the fluorine-18 labeled 4-[(3,4-dichloro-6-fluorophenyl)amino]-7-[3-(4-morpholinyl)propoxy]-6-nitroquinazoline is further reduced to the corresponding aminoquinazoline and reacted with α-chloroacetyl chloride or α-methoxyacetyl chloride as described to yield the final fluorine-18 labeled products.

### Synthesis of fluorine-18 Labeled [4-(phenylamino)quinazoline-6-yl]amides Substituted by a Leaving Group at the α Position—General Procedure II

A pre-selected diamino benzene is reacted with 4-chloro-6-nitroquinazoline, to yield the corresponding 4-(aminoaniline)-6-nitroquinazoline, which is further reacted with 3 equivalents of methyl trifluoromethylsulfonate, to yield the quaternary ammonioum salt of the above 4-(aminoaniline)-6-nitroquinazoline. The queaternary ammonium salt is then reacted with the Kryptofix®2.2.2—potassium ¹⁸F-fluoride—DMSO solution described above, to produce a fluorine-18 labeled 4-[(fluorophenyl)amino]-6-nitroquinazoline, which is thereafter reduced to obtain the amine derivative thereof, and is further reacted with a reactive carboxylic derivative as described herein.

Base on the general procedure II described hereinabove, fluorine-18 labeled of N-{4-[(4,5-Dichloro-2-fluoro-phenyl)amino]-quinazolin-6-yl}-2-chloroacetamide Fluorine-18 labeled Compound 5) and fluorine-18 labeled of N-{4-[(4,5-Dichloro-2-fluoro-phenyl)amino]-quinazolin-6-yl}-2-methoxyacetamide (Fluorine-18 labeled Compound 6) can be synthesized.

### Synthesis of iodine-123 Labeled, iodine-124 Labeled and iodine-131 Labeled N-{4-[(iodophenyl)amino]quinazolin-6-yl}amides Substituted by a Leaving Group at the α Position—General Procedure

3-Bromoaniline is coupled with 4-chloro-6-nitroquinazoline, to produce 4-[(3-bromophenyl)amino]-6-nitroquinazoline, which is reduced thereafter to the corresponding 6-aminoquinazoline, as is described hereinabove. The 4-[(3-bromophenyl)amino]-6-aminoquinazoline is then reacted with bistributyltin, using tetrakis(triphenylphosphine)palladium in triethylamine solution as the reaction catalyst. The stanylated quinazoline is then reacted with iodine-123, iodine-124 or iodine-131, in the presence of an oxidizing agent, to produce iodine-123 labeled, iodine-124 or iodine-131 labeled 4-[(3-iodophenyl)amino]-6-aminoquinazoline, which is further reacted a reactive carboxylic derivative (e.g., α-chloroacetyl chloride or α-methoxyacetyl chloride) as described, to yield the final iodine-123 labeled, iodine-124 labeled or iodine-131 labeled product.

### Synthesis of iodine-124 labeled 6-amino-4-[(3-iodophenyl)amino]-quinazoline

6-Amino-4-[(3-bromophenyl)-amino]-quinazoline (300 mg, 0.95 mmol, prepared as described hereinabove) was dissolved in dry THF (20 ml), and (SnBu₃)₂ (1.92 ml, 3.78 mmol) was added, followed by the addition of Pd(PPh₃)₄ (547.8 mg, 0.474 mmol) in dry THF (0.5 ml). The mixture was refluxed for 16 hours, and the solvent was thereafter evaporated. The crude product was purified over an aluminium oxide 90 column (70-230 mesh), using a mixture of 20:80 hexane:dichloromethane followed by 100% dichloromethane as eluents, to yield 6-amino-4-[(3-tributyltinphenyl)amino]-quinazoline (85 mg, 20%).

MS (m/z): 527 (M+2H)⁺;

¹H-NMR (CDCl₃): δ=8.592 (1H, s), 7.75 (1H, d, J=8.7 Hz), 7.64 (2H, m), 7.58 (1H, m), 7.47 (3H, m), 1.567 (6H, mt), 1.308 (6H, mt), 1.077 (6H, t, J=5.7 Hz), 0.919 (9H, t, J=7.2);

HPLC conditions: Normal-Phase analytical column, 100% acetonitrile, flow=1.0 ml/minute; Rₜ.=13.59 minutes.

The obtained 6-amino-4-[(3-tributyltinphenyl)amino]-quinazoline (4 mg) was placed in a conical vial, EtOH (1.2 ml) was added, followed by addition of 0.1 M [¹²⁴I] NaI (1 ml). 0.1 N HCl (1 ml) and Chloramine-T (1 mg/ml) (1 ml) were added, and the vial was sealed. The reaction was stirred at room temperature for 15 minutes, and thereafter sodium metabisulfite (200 mg/ml) (3 ml), a saturated solution of NaHCO₃ (6 ml) and saline solution (6 ml) were added. The aqueous solution was then vortexed, and loaded onto a C18 Sep-pak. The column was rinsed with water (2.5 ml), dried under nitrogen for 10 minutes, and the product was eluted with dry THF (4 ml). The THF solution was dried with Na₂SO₄, filtered through 0.45μ filter into a v-vial, and was used without any further treatment for the next step. The purity of the product was analyzed by a reversed-phase C18 analytical column (10 μm, 300×3.9 mm), eluted with 55% acetate buffer/45% acetonitrile, flow=1.0 ml/minute; Rₜ.=8.3 minutes.

The radiochemical yield of this step was measured by evaporating the THF solution, to a volume of 200 μl, and injecting the remaining solution onto a reversed-phase C18 preparative column.

The average radiochemical yield of the product was 50% (n=7).

HPLC conditions: C18 preparative column, eluted with 60% acetate buffer/40% acetonitrile, flow=3.0 ml/minute; Rₜ.=10.6 minutes.

### Synthesis of Iodine-124 Labeled N-{4-[(3-iodophenyl)amino]-quinazoline-6-yl}-2-methoxyacetamide (Iodine-124 Labeled Compound 4)

A THF solution of the iodine-124 labeled 6-amino-4-[(3-iodophenyl)amino]-quinazoline, obtained as described hereinabove (4 ml) was cooled to 0° C. for 10 minutes, and methoxyacetyl chloride (200 μl) in dry THF (300 μl) was added thereto. The reaction mixture was stirred for 30-40 minutes at 0° C. A mixture of ACN:H₂O (1:1) (200 μl) was added, and the solution was evaporated under nitrogen, while being cooled in an iced-water bath, to a volume of 400 μl. The crude product was purified using an HPLC reversed-phase C18 preparative column to yield the iodine-124 labeled product, with an overall radiochemical yield of 28%, specific activity of >6 Ci/mmol (the system detection limit) and 99% radiochemical purity (n=4).

HPLC conditions: C18 preparative column, 60% acetate buffer/40% acetonitrile, flow=4.0 ml/minute; Rₜ=22.31 minutes.;

HPLC conditions: C18 analytical column, 55% acetate buffer/45% acetonitrile, flow=1.0 ml/minute; Rₜ=10.78 minutes.

### Synthesis of Iodine-124 Labeled N-{4-[(3-iodophenyl)amino]-quinazoline-6-yl}-2-chloroacetamide (Iodine-124 Labeled Compound 3)

The iodine-124 labeled Compound 3 was prepared as described hereinabove for the iodine-124 labeled Compound 4, by reacting the iodine-124 labeled 6-amino-4-[(3-iodophenyl)amino]-quinazoline with chloroacetyl chloride (200 μl) in dry THF (300 μl). The iodine-124 labeled product was obtained with an overall radiochemical yield of 36% specific activity of >6 Ci/mmol (the system detection limit) and 99% radiochemical purity (n=4).

HPLC conditions: C-18 analytical column, 55% acetate buffer/45% acetonitrile, flow=1.0 ml/minute; Rₜ=13.16 minutes;

HPLC conditions: C18 preparative column, 55% acetate buffer/45% acetonitrile, flow=3.0 ml/minute; Rₜ=20.39 minutes;

HPLC conditions: C18 analytical column, 55% acetate buffer/45% acetonitrile, flow=1.0 ml/minute; Rₜ=13.16 minutes.

### Synthesis of Iodine-123 Labeled, Iodine-124 Labeled and Iodine-131 Labeled N-{4-[(3-iodophenyl)amino]-7-[3-(4-morpholinyl)propoxy]quinazoline-6-yl}2-chloro/2-methoxyacetamide (Iodine-123, Iodine-124 and Iodine-131 Labeled Morpholino-Substituted Compounds 3 and 4)

3-Bromoaniline is coupled with 4-chloro-7-fluoro-6-nitroquinazoline, to produce 4-[(3-bromophenyl) amino]-7-fluoro-6-nitroquinazoline, which is reacted thereafter with the sodium salt of 3-(4-morpholinyl)-1-propanol, as described hereinabove, to produce 4-[(3-bromophenyl)amino]-7-[3-(4-morpholinyl)propoxy]-6-nitroquinazoline. The morpholino-substituted 6-nitroquinazoline is then reduced to the corresponding 6-aminoquinazoline, which is further reacted with bistributyltin, iodine-123, iodine-124 or iodine-131 and α-methoxy- or α-chloro-acetyl chloride as described herein, as described hereinabove, to yield the final iodine-123 labeled, iodine-124 labeled or iodine-131 labeled products.

### Synthesis of Bromine-76 Labeled and Bromine-77 Labeled N-{4-[(bromophenyl)amino]quinazolin-6-yl}amides Substituted by a Leaving Group at the α Position—General Procedure

Bromoaniline is coupled with 4-chloro-6-nitroquinazoline, to produce 4-[(bromophenyl)amino]-6-nitroquinazoline, which is reduced thereafter to the corresponding 6-aminoquinazoline. The 4-[(bromophenyl)amino]-6-aminoquinazoline is then reacted with bistributyltin, using tetrakis(triphenylphosphine)palladium in THF solution as the reaction catalyst, as is detailed hereinabove. The stanylated quinazoline is then reacted with bromine-76 or bromine-77, in the presence of an oxidizing agent, to produce bromine-76 labeled or bromine-77 labeled 4-[(bromophenyl)amino]-6-aminoquinazoline, which is further reacted with a reactive carboxylic derivative (e.g., α-chloroacetyl chloride or α-methoxyacetyl chloride) as described, to yield the final bromine-76 labeled or bromine-77 labeled product.

### Synthesis of Bromine-76/Bromine-77 Labeled N-{4-[(3-bromophenyl)amino]quinazolin-6-yl}-2-chloro/2-methoxyacetamide (Bromine-76 Labeled/Bromine-77 Labeled Compounds 1 and 2)

3-Bromoaniline was coupled with 4-chloro-6-nitroquinazoline, to produce 4-[(3-bromophenyl)amino]-6-nitroquinazoline, which was reduced thereafter to the corresponding 6-aminoquinazoline, as is described hereinabove. The 4-[(3-bromophenyl)amino]-6-aminoquinazoline was then reacted with bistributyltin, using tetrakis(triphenylphosphine)palladium in THF solution as the reaction catalyst, as is detailed hereinabove. The stanylated quinazoline is then reacted with bromine-76 or bromine-77, in the presence of an oxidizing agent, to produce bromine-76 labeled or bromine-77 labeled 4-[(bromophenyl)amino]-6-aminoquinazoline, which is further reacted with α-chloroacetyl chloride or α-methoxyacetyl chloride as described, to yield the final bromine-76 labeled or bromine-77 labeled products.

### Synthesis of Bromine-76 Labeled and Bromine-77 Labeled N-{4-[(3-bromophenyl)amino]-7-[3-(4-morpholinyl)propoxy]quinazoline-6-yl}-2-chloro/2-methoxyacetamide (Bromine-76 and Bromine-77 Labeled Morpholino-Substituted Compounds 1 and 2)

3-Bromoaniline is coupled with 4-chloro-7-fluoro-6-nitroquinazoline, to produce 4-[(3-bromophenyl) amino]-7-fluoro-6-nitroquinazoline, which is reacted thereafter with the sodium salt of 3-(4-morpholinyl)-1-propanol, as described hereinabove, to produce 4-[(3-bromophenyl)amino]-7-[3-(4-morpholinyl)propoxy]-6-nitroquinazoline. The morpholino-substituted 6-nitroquinazoline is then reduced to the corresponding 6-aminoquinazoline, which is further reacted with bistributyltin, bromine-76 or bromine-77 and α-chloroacetyl chloride or α-methoxyacetyl chloride, as described hereinabove, to yield the final bromine-76 labeled or bromine-77 labeled products.

### Synthesis of N-[4-(phenylamino)quinazoline-6-yl]amides Substituted by a Leaving Group and by a Radioactive Carbon, Radioactive Fluorine, Radioactive Bromine and/or Radioactive Iodine Labeled Group at the α Position—General Procedure

A reactive carboxylic derivative, such as acetyl chloride substituted at the α position by a leaving group and by one or more radiolabeled (e.g., fluorine-18, bromine-76, bromine-77, iodine-123, iodine-124, iodine-131 and/or carbon-11 labeled) group(s) is prepared according to known procedures.

A 6-Amino-4-(phenylamino)quinazoline is prepared as described hereinabove and thereafter reacted with the radiolabeled reactive carboxylic derivative, at 0° C. in THF, in the presence of a chemically reactive base such as tertiary amine, to give the final product.

In Vitro Activity Assays:

Primary antibodies were obtained as follows: PY20 anti phosphotyrosine (diluted 1:2,000) from Santa Cruz Biotechnology Inc. 4G10 anti phosphotyrosine antibody (1:100 dilution) was produced from Su4G10 hybridoma cells. Horseradish peroxidase-conjugated anti-mouse IgG (1:10,000 dilution) was obtained from Jackson Immuno Research Growth factors. Human, recombinant EGF and PDGF_{ββ} were purchased from Sigma-Aldrich, Inc.

NIH3T3 cells transformed with either the EGFR (DHER14 cells), with the HER1-HER2 chimera (CSH12 cells) or with the PDGFR (NIH/PDGFR cells), decribed by Lee et al.; 1989, Honegger et al., 1988; and Shawver et al. 1997, and A431 human epidermoid vulval carcinoma cells were grown in Dulbecco's modified Eagle's medium (DMEM) (Biological industries, Kibbuts Beit Haemek, Israel) supplemented with 10% fetal calf serum and antibiotics (penicillin 105 units/liter, streptomycin 100 mg/liter) at 37° C. in 5% CO₂.

Chalenge Reactions with Reduced Glutathione:

Standard solutions were prepared by dissolving compound 5, Compound 6 and {4-[(3,4-dichloro-6-fluorophenyl)amino]quinazol ine-6-yl}acrylamide (0.0146 mmol) in 1.75 ml of THF: MeOH (1:2) and glutathione (18 mg, 0.0586 mmol) in 0.5 ml of water. A 300 μL aliquot of the quinazoline standard solution (2.5 μmol) was diluted with 689 μL of THF:MeOH:H₂O (1:2:1), after which 11 μL (1.25 μmol) of glutathione solution and 5.22 μL (30 μmol) of N,N′-diisopropylethylamine were added. Conversions of the quinazolines and formation of conjugates at different time points were measured by HPLC using RP (3.9×300 mm) column (mobile phase of acetonirile and acetate buffer 0.1 M (2:3) at a flow rate of 1 ml/min was used herein). The glutathione conjugates were further detected by MS.

Autophosphorylation Inhibition Experiments in A431 Cell Lysate:

EGFR-TK source: A431 cells were grown in 14 cm petri dishes to about 90% confluence. The dishes were then washed twice with cold phosphate buffered saline (PBS) Ph 7.4, placed on ice, and 3.25 ml cold, freshly prepared lysis buffer (50 mM N-2-hydroxyethylpiperazine-N′-2-ethanesulfonic acid (HEPES) buffer pH 7.4, 150 mM NaCl, 1% Triton X-100, 10% glycerol, 1 mM 4-(2-aminoethyl)benzenesulfonylfluoride hydrochloride (AEBSF), 1 μg/ml aprotinin, 300 μg/ml benzamidine, 10 μg/ml leupeptin, 10 μg/ml soy-trypsin inhibitor) was added for 10 minutes. The cells were scraped from the plates with a rubber policeman, homogenized with a dounce homogenizer, and centrifuged (Sorvall centrifuge, rotor 5, 10,000 rpm, 10 minutes, 4° C.). The supernatant, which contained the EGFR, was collected and frozen at −70° C. in aliquots.

ELISA assay: EGFR-TK autophosphorylation IC₅₀ values were obtained by means of an ELISA assay. All the following incubations were performed at room temperature and with constant shaking. After each step the plate was washed with 200 μl water (×4) and 200 μl TBST buffer (×1). The final volume for each well was 150 μl.

A Corning 96 well ELISA plate was coated with monoclonal anti EGFR antibody mAb108 (Sugen Inc.), diluted in PBS (pH 8.5), and kept overnight at 4° C. The total mAb108 content per well was 0.75 μg. After removing the unbound mAb108, the plate was washed and PBS containing 5% milk (1% fat) was added for the blocking (30 minutes).

One aliquot of A431 cell lysate was thawed, diluted with PBS pH 7.4 and added to the plate at a final total protein concentration of 10 μg/well.

After 30 minutes, various concentrations of each inhibitor were added, and for each case one well was left as a zero-inhibition control (no inhibitor) and one well was left as a zero-EGFR-TK control (no lysate). The inhibitors were diluted in TBS/DMSO and the final concentration of DMSO was 0.05% in each well (including the controls).

After additional 30 minutes, and without washing the plate, ATP/MnCl₂ solution was added in each well. The final concentration was 5 μM ATP/5 mM MnCl₂. In this step the temperature was kept at 26° C. and the plate was under constant shaking. The incubation with ATP/MnCl₂ was for 5 minutes.

Then, to stop the phosphorylation reaction, EDTA was added (pH 8, final concentration in each well 100 mM) and after 10 minutes the plate was washed.

Afterward, polyclonal anti-phosphotyrosine serum (Sugen, Inc.) was added (dilution of antibody in TBST containing 5% milk). The incubation was for 45 minutes.

For the colorimetric detection of phosphotyrosine in EGFR-TK, TAGO anti-rabbit peroxidase conjugate antibody (Sugen, Inc.) was added in TBST/5% milk solution (45 minutes).

After washing, the colorimetric reaction was performed by adding 100 μl/well ABTS/H₂O₂ in citrate-phosphate buffer pH 4.0 (7.5 mg 2-2′-azino-bis(3-ethylbenzethiazoline-6-sulfonic acid) (ABTS), 2 μL 30% H₂O₂, 15 mμ citrate-phosphate buffer pH 4.0). After 5-10 minutes the plate was read on Dynaytec MR 5000 ELISA reader at 405 nm.

The analysis of the data was performed using GraphPad Prism, version 2.01 (GraphPad Software, Inc.).

Autophosphorylation Inhibition Experiments in Intact A431 Cells:

A431 cells (5×10⁵) were seeded in 6-well plates and grown for 24 hours to about 90% confluence in DMEM (high glucose) containing 10% fetal calf serum (FCS) and antibiotics at 37° C. The cells were then exposed to serum-free medium, at 37° C., for 18 hours.

Irreversibility assay: Variable concentrations of the inhibitor, ranging from 0.05 nM to 50 nM, were added to A431 cells for 1 hour incubation. The medium was replaced thereafter with an inhibitor/FCS-free medium and the cells were divided into two groups: cells of the first group were immediately stimulated with EGF (20 ng/ml) for 5 minutes and then washed with PBS, while cells of the second group were incubated for additional 8 hours, at 37° C. During the 8 hours period, the medium was changed three times (after 2, 4 and 8 hours). After the post-incubation period, the cells of the second group were stimulated with EGF (20 ng/ml) for 5 minutes and then washed with PBS. Whole-cell lysates were obtained by scraping the cells into the well with 0.4 ml of Leammli buffer (10% glycerol, 2% sodium dodecyl sulfate, 5% b-mercaptoethanol, 62.5 mM Tris pH 6.8) that contained 0.001% bromophenol blue, and boiling for 5 minutes.

Selective-inhibition assay: CSH12, DHER14 and NIHPDGFR cells, expressing either the HER1-HER2 chimera, EGFR or PDGFR, respectively, were used for the determination of inhibitory selectivity. Cells (7.5×10⁴) were grown in 6-well plates (35 mm diameter, Nalge Nunc) for 24 hours and then incubated in 0.25% FCS-containing medium for an additional 24 hours to about 90% confluence. Duplicate sets of cells were treated with the tested compounds at varying concentrations for 1 hour. The final concentration of the vehicle in the medium was 0.05% DMSO, 0.1% EtOH. After removal of the inhibitor from the medium, PBS wash (×2) and addition of 0.25% serum-containing medium to the wells, the cells were stimulated with either 20 ng/mL human EGF for 5 minutes (CSH12 and DHER14 cells) or 50 ng/ml human PDGF_{ββ} for 10 minutes (NIHPDGFR cells) at 37° C. Following the stimulation with the growth factor, the cells were washed with cold PBS. Cell extracts were made by adding 0.4 ml boiling Laemmli buffer (10% glycerol, 2% sodium dodecyl sulfate, 5%-mercaptoethanol, 62.5 mM Tris.HCl pH 6.8) containing 0.001% bromophenol blue to the cells, scraping the xells with a rubber policeman and heating to 100° C. for 10 minutes. For each compound, at least two different assays with similar results were performed. Each experiment was carried out in duplicates.

Western Blot Analysis:

Identical protein amounts from each lysate sample were loaded onto polyacrylamide gel (6% or 10%), separated by electrophoresis (Hoefer Pharmacia Biotech Inc., San Francisco, USA) and transferred to nitrocellulose membrane (power supply: EPS 500/400, Amersham Pharmacia Biotech; nitrocellulose extra blotting membranes: Sartorius AG, Goettingen, Germany). A standard high molecular weight solution was loaded as a reference. For visualization of molecular weight bands, the membrane was immersed in Ponceau reagent (0.05% Ponceau, 5% acetic acid) for a few minutes, and then washed twice with TTN (10 mM Tris pH 7.4, 0.2% TWEEN 20, 170 mM NaCl) and once with water. The membrane was blocked overnight in TTN containing 5% milk (1% fat) (blocking TTN) and incubated for 90 minutes with PY20 antiphosphotyrosine antibody (Santa Cruz Biotechnology Inc., Santa Cruz, USA) diluted 1:2,000 in blocking TTN. The membrane was then washed with TTN (3×5 minutes), incubated for 90 minutes with a horseradish peroxidase-conjugated secondary antibody (Goat anti-mouse IgG H+L, Jackson ImResearch Laboratories, Inc., diluted 1:10,000 in blocking TTN), and finally washed again with TTN (3×5 minutes). The membrane was incubated in a luminol-based solution (1 minute, 0.1 M Tris pH 8.5, 250 μM luminol, 400 μM p-cumaric acid, 0.033% H₂O₂) and visualized using chemiluminescent detection.

Quantification of the EGFR-P (protein) bands density obtained was performed using Adobe Photoshop 5.0ME and NIH image 1.16/ppc programs.

### Experimental Results

Chemical and Radio Syntheses:

In a quest for novel irreversible EGFR-TK inhibitors with improved in vivo performance, as compared with the presently known inhibitors, various N-{4-[(phenyl amino)quinazoline-2-yl]}acetamides, all substituted by a leaving group at the α position of the acetamide, were synthesized.

Thus, Compounds 1-6 were prepared as exemplary compounds for other N-{4-[(phenylamino)quinazoline-2-yl]}acetamides substituted by one or more leaving groups at the a position. This class of compounds is prepared by reacting an aniline derivative with 4-chloroquinazoline substituted by a reactive group, and reacting the obtained reactive product with a reactive carboxylic derivative substituted by a leaving group at the a position to produce the final compound.

As is shown in FIG. 2, Compounds 1-6 were prepared by reacting an anilne derivative with 4-chloro-6-nitroquinazoline (Compound 7) to produce compound 8, reducing the nitro group of compound 8 to the amino group, using an ethanolic solution of hydrazine hydrate and Raney®Nickel as described, to produce compound 9 and reacting compound 9 with either α-chloroacetyl chloride or α-methoxyacetyl chloride as described, at 0° C., to produce the final product.

In order to enhance the biological availability of the compounds of the present invention, derivatives of N-{4-[(phenylamino)quinazoline-2-yl]}acetamides substituted by a leaving group at the a position, which are further substituted by a morpholino or piperazino group, preferably at position 7 (e.g., 7-morpholino-substituted Compounds 1-6), can also be prepared according to known procedures (see, Smaill et al., 2000 and U.S. Patent Application No. 20020128553), as described hereinabove.

The novel irreversible EGFR-TK inhibitors of the present invention can be radiolabeled, to thereby produce radiolabeled irreversible EGFR-TK inhibitors for use in radioimaging and radiotherapy. As is detailed hereinabove, by selecting the appropriate aniline derivative, N-{4-[(phenylamino)quinazoline-2-yl]}acetamides substituted by a leaving group at the a position, and optionally substituted by a morpholino group at the quinazoline ring, radiolabeled by radioactive iodine, radioactive bromine, or radioactive fluorine, can be prepared, using the following optional radiolabeling strategies:

The first strategy involves the use of fluorine-18 in order to label the aniline moiety at position 6 thereof. Radiolabeling with Fluorine-18 can be performed using known procedures (Mishani et al., 1997, U.S. Pat. Nos. 6,126,917 and 6,562,319) or a newly developed automated radiosynthesis, which is based on a well-known nucleophilic substitution of tetramethyl-ammonium salts. A representative example of the latter, in which fluorine-18 labeled Compounds 5 and 6 are prepared, is described hereinabove and is further depicted in FIG. 3.

The second strategy involves the use of radioactive bromine (e.g., bromine-76 and bromine-77) or radioactive iodine (e.g., iodine-123, iodine-124 or iodine-131) in order to label the aniline moiety at position 3 thereof, using established radioiodination and radiobromination chemistry. As is shown in FIG. 4, 4-[(3-bromophenyl)amino]-6-nitroquinazoline is reacted with tributyltin, to produce the stanylated Compound 10, which is thereafter reacted with a radioactive oxidant, reduced to the corresponding aniline and reacted with α-chloroacetyl chloride or α-methoxyacetyl chloride to produce the radioactive bromine-labeled Compounds 1 and 2, or radioactive iodine-labeled Compounds 3 and 4.

As iodine-124 has recently become increasingly significant in PET diagnostic use and a potential therapeutic radionuclide, due to its radiocharacteristics (T_{1/2}=4.2 days, simultaneous positron emission and electron capture), preparation of an iodine-124 labeled irreversible EGFR inhibitor is highly desirable.

Hence, as representative examples of a radiolabeled irreversible EGFR-TK inhibitor, iodine-124 labeled Compounds 3 and 4 were prepared.

As is demonstrated hereinbelow, in the activity studies conducted with the novel compounds of the present invention, the 3,4-dichloro-6-fluorophenyl derivative Compound 5 was found to be a highly potent irreversible EGFR-TK inhibitor. Hence, fluorine-18 labeled Compounds 5 and 6, which may also serve as highly potent diagnostic tools, were prepared.

Alternatively, by selecting the appropriate carboxylic derivative, N-{4-[(phenylamino)quinazoline-2-yl]}acetamides substituted by a leaving group at the a position, radiolabeled by radioactive iodine, radioactive bromine, radioactive fluorine and/or radioactive carbon at the carboxylic side chain, can also be prepared, using a different strategy, which involves the use of a pre-radiolabeled reactive carboxylic derivative, as described hereinabove.

In Vitro Studies:

Chalenge Reactions with Reduced Gluthatione:

As discussed hereinabove, EGFR blockade by irreversible inhibitors is due to the nucleophilic attack of the sulfhydryl group of Cys-773 at the receptor's ATP binding pocket on the reactive chemical group of the EGFR targeted inhibitor. In order to evaluate the chemical reactivity of the novel irreversibly EGFR inhibitors decribed herein, the degree of reactivity of the inhibitors towards the sulfhydryl group of reduced glutathione (GSH) as a nucleophile was tested. Thus, Compound 5, Compound 6 and {4-[(3,4-dichloro-6-fluorophenyl)amino]quinazoline-6-yl}acrylamide were dissolved in THF: MeOH: H₂O (1:2:1), and were reacted at room temperature with half an equivalent of reduced glutathione in the presence of 12 equivalents of N,N-diisopropylethylamine. Identical aliquots of the reaction mixtures were taken at various time points and injected into reversed-phase HPLC in order to determine the conversion-rate of the various compounds into glutathione-conjugates. The characteristics of the products were determined using MS.

For each of the four reactions, a graph of product concentration as a function of time was plotted. A reaction rate constant of 5×10⁻⁸ was measured for Compound 5, of 7.0×10⁻⁵ was measured for Compound 6, and of 1.0×10⁻⁴ M/minute was measured for {4-[(3,4-dichloro-6-fluorophenyl)amino]quinazoline-6-yl}acrylamide.

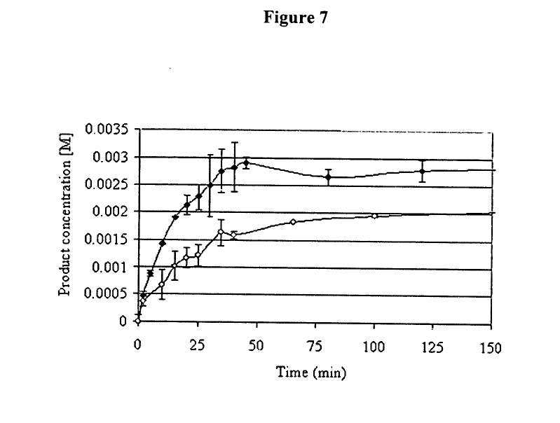

FIG. 7 presents the results obtained with Compound 5, compared with {4-[(3,4-dichloro-6-fluorophenyl)amino]quinazoline-6-yl}acrylamide.

A study of the reaction rate as a function of the temperature was also performed. Thus, the reaction ws performed at various temperatures ranging from 0° C. and 60° C. The following activation parameters were generated: For {4-[(3,4-dichloro-6-fluorophenyl)amino]quinazoline-6-yl}acrylamide: Ea=5.24 kCalmol⁻¹, ΔH^{#}_{25° C.}=4.64 kCalmol⁻¹ and ΔS^{#}_{25° C.}=−61.24 Calmol⁻¹K⁻¹. For compound 5: Ea=11.4 kCalmol⁻¹, ΔH^{#}_{25° C.}=10.80 kCalmol⁻¹ and ΔS^{#}_{25° C.}=−41.29 Calmol⁻¹K⁻¹.

For compound 6, the activation energies were too high, thus even at temperatures exceeding 100° C., no change in the reaction rate was obtained.

The reaction rate was calculated using the following rate equation:

$$\frac{- ⅆ [ {MLO}_{x} ]}{ⅆ t} = {k}_{obs} \times [ {MLO}_{x} ] \times [ GSH ]$$

k_{obs}=Ae^{-Ea/RT}

Where [ML0ₓ] and [GSH] represent the concentrations of the tested compound and of glutathione, respectively.

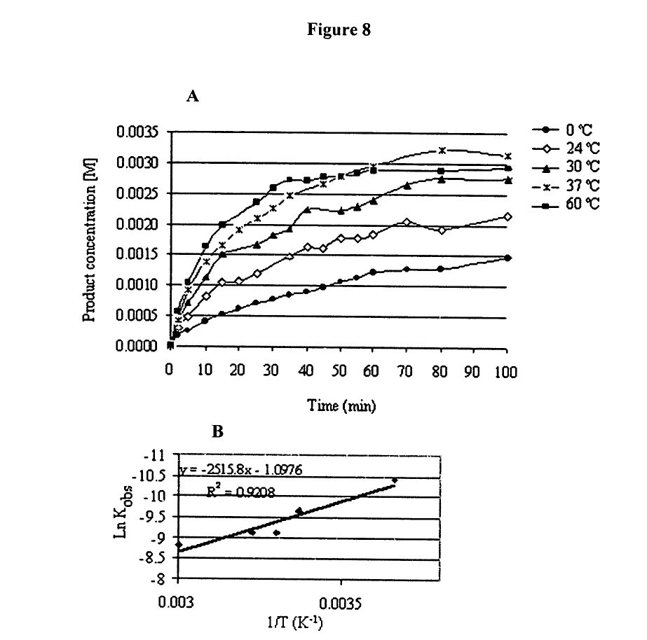

The results obtaind in this study are presented in FIGS. 8a and 8b.

Overall, the results of the challenge assay of the different groups of compounds with reduced glutathione demonstrated the improved chemical stability of the novel inhibitors described herein. Hence, the chloroacetamide Compound 5 was found to be less reactive than {4-[(3,4-dichloro-6-fluorophenyl)amino]quinazoline-6-yl}acrylamide, possessing a reaction rate constant of 7.0×10⁻⁵ M/minute. The methoxyacetamide Compound 6 was found to be far more stable towards the nucleophilic attack of the sulfhydryl group, possessing a reaction rate constant of 5.0×10⁻⁸ M/minute. Similar results were obtained while measuring the activation parameters and reaction rate. Due to the considerably higher activation energy of compound 6 a change in the reaction rate thereof with GSH could not be detected even at temperatures exceeding 100° C.

Autophosphorylation Inhibition:

EGFR-TK autophosphorylation IC₅₀ values were measured for Compounds 1-6 in order to determine their potential as therapeutic agents. The method employed an ELISA assay based on an anti-EGFR antibody. Since the measured compounds have an irreversible inhibition kinetic, the IC₅₀ values thereof are apparent values, which were calculated using a non-linear regression fit to a variable slope sigmoidal dose response curve. The ELISA assay was performed twice and the apparent IC₅₀ averages were determined from four independent dose-response curves. The IC₅₀ values obtained for Compounds 1-6 are presented in Table 1 below, and are compared with the IC₅₀ values obtained with the known irreversible EGFR-TK inhibitors of the anilinoquinazoline family, N-{4-[(3,4-dichloro-6-fluorophenyl)amino]quinazoline-6-yl}acrylamide and N-{4-[(3-bromo)amino]quinazoline-6-yl}-4-(methylamino)-2-butenamide, which are referred to in Table 1 as Compound A and Compound B, respectively. Compound A is characterized by high affinity toward EGFR, whereas Compound B is characterized by high ability to form irreversible binding to EGFR.

As is shown in Table 1, the obtained IC₅₀ values indicate that the compounds of the present invention, which are substituted by a α-chloroacetamide side chain, namely Compounds 1, 3 and 5, exert high affinities toward EGFR. The compounds substituted by a α-methoxyacetamide side chain, namely Compounds 2, 4 and 6, are somewhat less potent, as compared with both the α-chloroacetamide substituted compounds and Compound A. However, the IC₅₀ values obtained for these compounds indicate that these compounds may serve as good candidates for both therapy and diagnosis.

TABLE 1

| | | Intact A431 cells | |
|---|---|---|---|
| | | IC₅₀ range | IC₅₀ range |
| | | (1 hr post | (8 hr post |
| | A431 lysate | incubation with | incubation with |
| Structure | IC₅₀ app | inhibitor) | inhibitor) |

| | | | | | | |
|---|---|---|---|---|---|---|
| 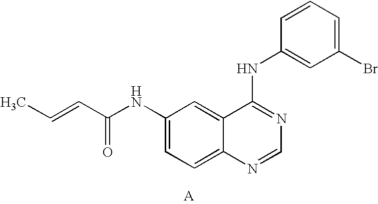 | 161 +/− 31 | nM | <<1 | μM | approx. 10 | μM |
| 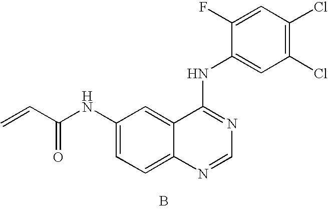 | 0.037 | nM | 5-50 | nM | 5-50 | nM |
| 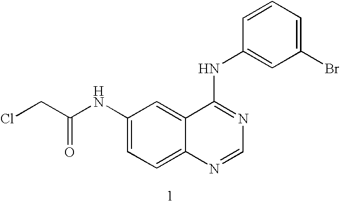 | 20.0 +/− 11.6 | nM | 10-50 | nM | 10-50 | nM |
| 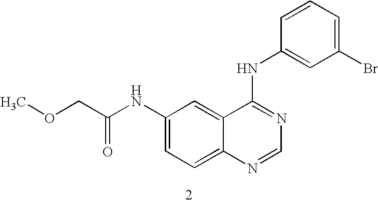 | 60 +/− 12 | nM | 1.3-26.7 | μM | 100-160 | μM |
| 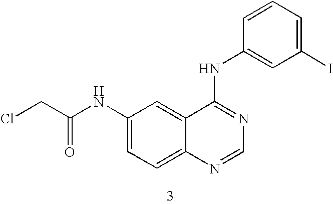 | 17.8 +/− 10.4 | nM | 4-10 | nM | 10-50 | nM |
| 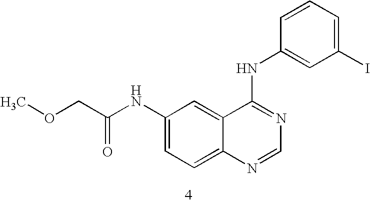 | 65 +/− 15 | nM | 1-20 | μM | >80 | μM |
| 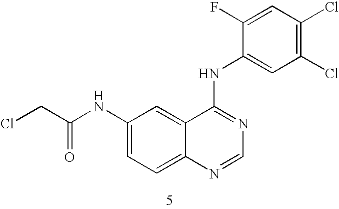 | 5.55 +/− 1.13 | nM | 1-20 | nM | 1-20 | nM |
| 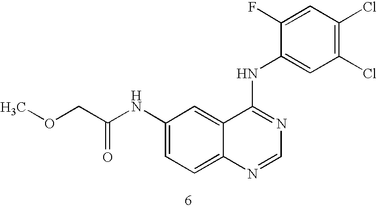 | 113 +/− 18 | nM | 1-10 | μM | 10-50 | μM |

The irreversible nature of Compounds 1-6 EGFR-TK binding were evaluated by measuring the inhibition of EGFR-TK autophosphorylation in intact A431 cell line. The results obtained in these studies are also presented in Table 1 above.

In order to demonstrate the irreversibility of the binding of Compounds 1-6 to the receptor, the cells were incubated with variable inhibitor concentrations for 1 hour. After the incubation, the media was replaced with inhibitor/FCS-free media and the inhibition effect was measured either immediately thereafter or after 8 hours post incubation. As previously described (see, for example, Smaill et al., 1999), 80% or more inhibition, achieved after 8 hours, indicate that the compound is irreversible, while 20-80% inhibition classify the compound as “partially irreversible”.

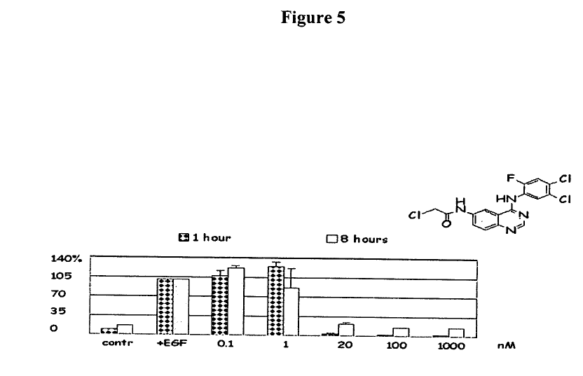

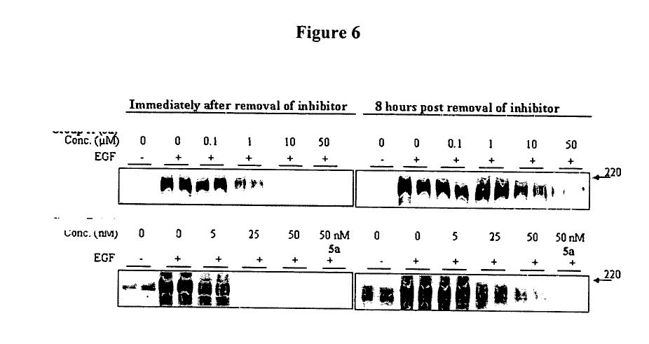

As is presented in Table 1 and is further shown in FIGS. 5 and 6, Compounds 1, 3 and 5 of the present invention, which are substituted by an α-chloroacetamide group, retained the irreversible binding nature to the receptor. Eight hours post incubation, 50% inhibition was already achieved with an inhibitor concentration of approximately 10-50 nM, reflecting the irreversible effect of these inhibitors, which is most likely, due to covalent binding at the ATP binding site.

Compounds 2, 4 and 6, which are substituted by the more chemically stable α-methoxyacetamide group, exerted a partial irreversible binding to the receptor at higher inhibitors concentrations.

These results demonstrate for the first time that a chain of 4 atoms attached to the quinazoline moiety is not an essential feature for irreversible binding, as was previously suggested (see, Smaill et al., 1999 and 2000). Structurally, a chain of 3 atoms is sufficient to achieve covalent binding at the receptor-binding pocket.

Selectivity:

Binding selectivity of a PET probe to its molecular target is a significant determinant in its ability to serve as a high-quality imaging agent. In order to characterize the degree of specificity of the compounds in inhibiting the EGFR, Compound 5 and 6 were tested out in a cellular assay, similar to the assay performed with A431 cells, as described above. In brief, DHER14, CSH12 and NIH/PDGFR cells, expressing EGFR, EGFR-HER2 chimera or PDGFR, respectively, were incubated with the tested inhibitor for one hour. Following removal of the inhibitor from the medium and stimulation with the appropriate growth factor, the cells were harvested, and the extent of inhibition was evaluated by measuring the phosphotyrosine content of the receptor in a Western blot analysis.

The obtained data are presented in Table 2 below and reveal that the tested compounds bear no inhibitory effect upon the PDGFR (IC₅₀>1 μM). Nonetheless, the inhibitory profile with respect to the kinase domain of HER2 and EGFR was similar to that observed in A431 cells: Compound 6 was far more potent in inhibiting both EGFR/c-ErbB1 and c-ErbB2.

These preliminary selectivity stdies thus demonstrate a good selectivity profile of the novel inhibitors described herein, and particularly of the methoxyacetamide family, indicated by more than three-fold higher inhibitory concentrations for the PDGFR, as compared with the erbB-1 and 2 kinase domains.

TABLE 2

| | IC₅₀ value (nM) | | |
|---|---|---|---|
| Structure | DHER14 | CSH12 | NIHPDGFR |
| Compound 6 | >250 | >500 | >1,000 |
| Compound 5 | 5-15 | 25-50 | >1,000 |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### LIST OF REFERENCES CITED

Ackermann, U., Tochon-Dabguy, H., Nice, E., Nerrie, M., Young, K., Sachinidis, J., Scott, A. (2003). Synthesis, radiolabelling and biological evaluation of derivatives of the P210BCR-ABL tyrosine kinase inhibitor AG957, J Label Compounds Radiopharm 46 S118.
Arteaga, C. L., Ramsey, T. T., Shawver, L. K., and Guyer, C. A. (1997). Unliganded epidermal growth factor receptor dimerization induced by direct interaction of quinazolines with the ATP binding site. J Biol Chem 272, 23, 247-54.
Artega, C. L., (2001). The epidermal growth factor receptor: from mutant oncogene in nonhuman cancers to therapeutic target in human neoplasia, J Clin Oncol 19 32-40.
Baselga, J., and Averbuch, S. D. (2000). ZD1839 (‘Iressa’) as an anticancer agent. Drugs 60 Suppl 1, 33-40; discussion 41-2.
Ben-David, I., Rozen, Y., Ortu, G., and Mishani, E. (2003). Radiosynthesis of ML03, a novel positron emission tomography biomarker for targeting epidermal growth factor receptor via the labeling synthon: [C-11]Acryloyl chloride. Appl. Rad. Isotp., 58 (2), 209-217.
Bonasera, T. A., Ortu, G., Rozen, Y., Krais, R., Freedman, N. M., Chisin, R., Gazit, A., Levitzki, A., and Mishani, E. (2001). Potential (18)F-labeled biomarkers for epidermal growth factor receptor tyrosine kinase. Nucl Med Biol 28, 359-74.
Ciardiello, F. (2000). Epidermal growth factor receptor tyrosine kinase inhibitors as anticancer agents. Drugs 60 Suppl 1, 25-32; discussion 41-2.
Elderfield, R. C., Williamson, T. A., Gensler, W. J., Kremer, C. B. (1947). J Org Chem 12 405-421.
Escobar, N. I.; Morales, A. M.; Ducongu, J.; Torres, I. C.; Fernandez, E.; Gomez, J. A. (1998). Pharmacokinetics, biodistribution and dosimetry of 99mTc-labeled anti-human epidermal growth factor receptor humanized monoclonal antibody R3 in rats. Nucl. Med. Biol. 25, 17-23.
Escobar, N. I.; Torres, L. A.; Morales, A.; Ramos, M.; Alvarez, I.; Perez, N.; Fraxedas, R.; Rodriguez, O.; Rodriguez, N.; Perez, R.; Lage, A.; Stabin, M. G. (1998). J. Nucl. Med. 39, 15-23.
Faaland, C. A., Mermelstein, F. H., Hayashi, J., and Laskin, J. D. (1991). Rapid uptake of tyrphostin into A431 human epidermoid cells is followed by delayed inhibition of epidermal growth factor (EGF)-stimulated EGF receptor tyrosine kinase activity. Mol Cell Biol 11, 2697-703.
Fry, D. W., Bridges, A. J., Denny, W. A., Doherty, A., Greis, K. D., Hicks, J. L., Hook, K. E., Keller, P. R., Leopold, W. R., Loo, J. A., McNamara, D. J., Nelson, J. M., Sherwood, V., Smaill, J. B., Trumpp-Kallmeyer, S., and Dobrusin, E. M. (1998). Specific, irreversible inactivation of the epidermal growth factor receptor and erbB2, by a new class of tyrosine kinase inhibitor. Proc Natl Acad Sci USA 95, 12022-7.
Fry, D. W., Kraker, A. J., McMichael, A., Ambroso, L. A., Nelson, J. M., Leopold, W. R., Connors, R. W., and Bridges, A. J. (1994). A specific inhibitor of the epidermal growth factor receptor tyrosine kinase. Science 265, 1093-5.
Gazit, A., Chen, J., App, H., McMahon, G., Hirth, P., Chen, I., and Levitzki, A. (1996). Tyrphostins IV—highly potent inhibitors of EGF receptor kinase. Structure-activity relationship study of 4-anilidoquinazolines. Bioorg Med Chem 4, 1203-7.
Gazit, A., Osherov, N., Gilon, C., and Levitzki, A. (1996). Tyrphostins. 6. Dimeric benzylidenemalononitrile tyrophostins: potent inhibitors of EGF receptor tyrosine kinase in vitro. J Med Chem 39, 4905-11.
Kesarios, N., Tochon-Danguy, H-J., Ackermann, U., Sachinidis, J., Lambert, J., Nerrie, M., Nice, E., Scott, A. (2003). Synthesis and radiolabelling of a tyrosine kinase inhibitor of erbB2/neu (HER2) receptors, J Label Compounds Radiopharm 46 S119.
Han, Y., Caday, C. G., Nanda, A., Cavenee, W. K., and Huang, H. J. (1996). Tyrphostin AG 1478 preferentially inhibits human glioma cells expressing truncated rather than wild-type epidermal growth factor receptors. Cancer Res 56, 3859-61.
Honegger, A.; Dull, T. J.; Szapary, D.; Komoriya, A.; Kris, R.; Ullrich, A.; Schlessinger, J. Kinetic parameters of the protein tyrosine kinase activity of EGF-receptor mutants with individually altered autophosphorylation sites. EMBO J, 1988, 7, 3053-3060.
John, C. S., Sega, T., Kinuya, s., Le, N., Jeong, J. M., Paik, C. H., Reba, R. C., Varma, V. M., MsFee, J. G. (1993). An improved synthesis of [¹²⁵I] N-(diethylaminoethyl)-4-iodobenzamide: a potential ligand for imaging malignant melanoma. Nucl Med Biol 20 75.
Johnstrom, P., Fredriksson, A., Thorell, J. O., Hassan, M., Kogner, P., Borgstrom, P., Ingvar, M., and Stone-Elander, S. (1997). Synthesis and in vivo biodistribution of tyrosine kinase inhibitor, [methoxy-¹¹C]PD 153035. J Label Compds Radiopharm 40, 377-379.
Lee, J.; Dull, T. J.; Lax, I.; Schlessinger, J.; Ullrich, A. HER2 cytoplasmic domain generates normal mitogenic and transforming signals in a chimeric receptor. EMBO J. 1989, 8, 167-73.
Levitzki, A.; Gazit, A. (1995). Science 267, 1782-1788.
Mulholland, G. K.; Winkle, W.; Mock, B. H.; Sledge, G. (1995). J Nucl. Med. 36 (supplement), 71P.
Mulholland, G. K.; Zheng, Q.-H.; Winkle, W. L.; Carlson, K. A. (1997). J. Nucl. Med. 38, 141P (abstract number 529).
Mishani, E., Cristel, M. E., Dence, C. S., McCarthy, T. J., Welch, M. J., (1997). Application of a novel phenylpiperazine formation reaction to the radiosynthesis of a model fluorine-18-labeled radiopharmaceutical (18FTFMPP). Nucl. Med. Biol., 24 (3), 269-73.
Mishani, E., Bonasera, T. A., Rozen, Y., Ortu, G., Gazit, A., and Levitzki, A. (1999). Fluorinated EGFR-TK inhibitors-based tracers for PET. J Labelled Cpd Radiopharm 42, S27-29.
Mishani, E., Bonasera, T. A., Rozen, Y., Ortu, G., Gazit, A., Levitzki, A. (2000). Novel Epidermal Growth Factor Receptor-Kinase Binding Compounds for Positron Emission Tomography. U.S. Pat. No. 6,126,917.
Mishani, E., Bonasera, T. A., Rozen, Y., Ortu, G., Gazit, A., Levitzki, A. (2000). Potential PET Biomarkers for Epidermal Growth Factor Receptor Tyrosine Kinase. 2000 International chemical congress of pacific basin societies. Honolulu, Hi.
Mishani, E., Ben-David, I., Rozen, Y., Ortu, G., and Leviztki, A. (2001). Carbon-11 labeled irreversible inhibitor for mapping epidermal growth factor receptor tyrosine kinase (EGFR-TK). J. Labelled Cpd. Radiopharm 44, S99-101.
Mishani, E., Abourbeh, G., Ortu, G., Rozen, Y., Ben-David, I., Froimovaci, S., Dissoki, S., Gazit, A., Levitzki, A. (2003). Novel EGFR irreversible tyrosine kinase inhibitor candidates for the diagnostic and therapeutic treatment of cancer, J Label Compounds Radiopharm 46 S115.
Mishani, E., Abourbeh, G., Rozen, Y., Laki, D., Levitzki, A. (2003). Carbon-11 labeling of 4-dimethylamino-but-2-enoic acid [4-(phenylamino)-quinazoline-6-yl]-amides. A new class of EGFR irreversible inhibitors, J Label Compounds Radiopharm 46 S2.
Miyaji, K., Tani, E., Shindo, H., Nakano, A., and Tokunaga, T. (1994). Effect of tyrphostin on cell growth and tyrosine kinase activity of epidermal growth factor receptor in human gliomas. J Neurosurg 81, 411-9.
Nelson, J. M., and Fry, D. W. (1997). Cytoskeletal and morphological changes associated with the specific suppression of the epidermal growth factor receptor tyrosine kinase activity in A431 human epidermoid carcinoma. Exp Cell Res 233, 383-90.
Ortu, G., Ben-David, I., Rozen, Y., Freedman, N. M. T., Chisin, R., Levitzki, A., and Mishani, E. (2002). Labeled EGFRTK irreversible inhibitor (ML03). In vitro and in vivo properties, potential as PET biomarker for cancer and feasibility as anticancer drug. Int J Cancer 101 (4), 360-370.
Raymond, E., Faivre, S., and Armand, J. P. (2000). Epidermal growth factor receptor tyrosine kinase as a target for anticancer therapy. Drugs 60 Suppl 1, 15-23; discussion 41-2.
Renhowe, P. A. (2001). Growth factor receptor kinases in cancer. Annual Reports in Medicinal Chemistry 36, 109-118.
C. Ritter, C. L. Arteaga, (2003). The epidermal growth factor receptor-tyrosine kinase: a promising therapeutic target in solid tumors, Semin Oncol 30 3-11.
Rowinsky, E. K. (2000). The pursuit of optimal outcomes in cancer therapy in a new age of rationally designed target-based anticancer agents. Drugs 60 Suppl 1, 1-14; discussion 41-2.
Salomon, D. S., Brandt, R., Ciardiello, F., and Normanno, N. (1995). Epidermal growth factor-related peptides and their receptors in human malignancies. Crit Rev Oncol Hematol 19, 183-232.
Seymour, L. K. (2001). Epidermal growth factor receptor as a target: recent developments in the search for effective new anti-cancer agents. Curr Drug Targets 2, 117-33.
Shawver, L. K.; Schwartz, D. P.; Mann, E.; Chen, H.; Tsai, J.; Chu, L.; Taylorson, L.; Longhi, M.; Meredith, S.; Germain, L.; Jacobs, J. S.; Tang, C.; Ullrich, A.; Berens, M. E.; Hersh, E.; McMahon, G.; Hirth, K. P.; Powell, T. J. Inhibition of platelet-derived growth factor-mediated signal transduction and tumor growth by N-[4-(trifluoromethyl)-phenyl]5-methylisoxazole-4-carboxamide. Clin Cancer Res. 1997, 3 (7), 1167-77.
Smaill, J. B., Palmer, B. D., Rewcastle, G. W., Denny, W. A., McNamara, D. J., Dobrusin, E. M., Bridges, A. J., Zhou, H., Showalter, H. D., Winters, R. T., Leopold, W. R., Fry, D. W., Nelson, J. M., Slintak, V., Elliot, W. L., Roberts, B. J., Vincent, P. W., and Patmore, S. J. (1999). Tyrosine kinase inhibitors. 15. 4-(Phenylamino)quinazoline and 4-(phenylamino)pyrido[d]pyrimidine acrylamides as irreversible inhibitors of the ATP binding site of the epidermal growth factor receptor. J Med Chem 42, 1803-15.
Smaill, J. B., Rewcastle, G. W., Loo, J. A., Greis, K. D., Chan, O. H., Reyner, E. L., Lipka, E., Showalter, H. D., Vincent, P. W., Elliott, W. L., and Denny, W. A. (2000). Tyrosine Kinase Inhibitors. 17. Irreversible Inhibitors of the Epidermal Growth Factor Receptor: 4-(Phenylamino)quinazoline- and 4-(Phenylamino)pyrido. J Med Chem 43, 1380-1397.
Tsou, H. R., Mamuya, N., Johnson, B. D., Reich, M. F., Gruber, B. C., Ye, F., Nilakantan, R., Shen, R., Discafani, C., DeBlanc, R., Davis, R., Koehn, F. E., Greenberger, L. M., Wang, Y. F., and Wissner, A. (2001). 6-Substituted-4-(3-bromophenylamino)quinazolines as putative irreversible inhibitors of the epidermal growth factor receptor (EGFR) and human epidermal growth factor receptor (HER-2) tyrosine kinases with enhanced antitumor activity. J Med Chem 44, 2719-34.
VanBrocklin, H. F., Dorff, P. N., Vasdev, N., O'Neil, J. P., Nandanan, E., Gibbs, A. R. (2003). Synthesis of 2′-,3′-and 4′-[F18]fluoroanilinoquinazoline, J Label Compounds Radiopharm 46 S117.
Voldborg, B. R., Damstrup, L., Spang-Thomsen, M., and Poulsen, H. S. (1997). Epidermal growth factor receptor (EGFR) and EGFR mutations, function and possible role in clinical trials. Ann Oncol 8, 1197-206.
Walsh, J. C., Maclean, D., Northrop, J., Padgett, H., Ysaguirre, T. (2003). A combinatorial strategy for the design and synthesis of 18F-labeled quinoline derivatives as kinase imaging agents, J Label Compounds Radiopharm 46 S48.
Yuste, F., Saldaña, M., Walls, F., (1982). Selective reduction of aromatic nitro compounds containing O- and N-benzyl groups with hydrazine and Raney nickel. Tet. Lett., 23 (2), 147-8.

What is claimed is:

1. A compound having the general Formula 1: 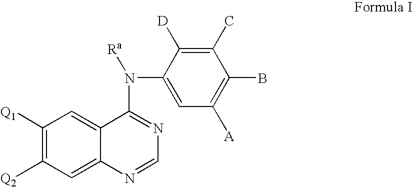
wherein:
Q1 is X—W(═Y)-Z and Q2 is selected from the group consisting of hydrogen, halogen, alkoxy, hydroxy, thiohydroxy, thioalkoxy, alkylamino and amino, or
Q1 is selected from the group consisting of hydrogen, halogen, alkoxy, hydroxy, thiohydroxy, thioalkoxy, alkylamino and amino and Q2 is X—W(═Y)-Z;
X is selected from the group consisting of —NR¹—, —O—, —NH—NR¹—, —O—NR¹—, NH—CHR¹—, —CHR¹—NH—, —CHR¹—O—, —O—CHR¹—, —CHR¹—CH₂— and —CHR¹—S— or absent;
W is carbon;
Y is selected from the group consisting of oxygen and sulfur;
Z is —CR²R³R⁴;
R^{a} is selected from the group consisting of hydrogen or alkyl having 1-8 carbon atoms;
A, B, C and D are each independently selected from the group consisting hydrogen and a first derivatizing group;
R¹ is selected from the group consisting of hydrogen, and substituted or non-substituted alkyl having 1-6 carbon atoms;
R² is a leaving group selected from the group consisting of halogen, alkoxy, aryloxy, thioalkoxy, thioaryloxy, azide, sulfinyl, sulfonyl, sulfonamide, phosphonyl, phosphinyl, carboxy and carbamyl; and
R³ and R⁴ are each independently selected from the group consisting of hydrogen and a second derivatizing group.

2. The compound of claim 1, wherein Q1 is X—W(═Y)-Z and Q2 is selected from the group consisting of hydrogen, halogen, alkoxy, hydroxy, thiohydroxy, thioalkoxy, alkylamino and amino.

3. The compound of claim 1, wherein X is said —NR¹— and Y is oxygen.

4. The compound of claim 3, wherein each of R¹, R³ and R⁴ is hydrogen.

5. The compound of claim 1, wherein R² is a leaving group selected from the group consisting of alkoxy and halogen.

6. The compound of claim 1, wherein D is fluorine.

7. The compound of claim 6, wherein A and B are each chlorine and C is hydrogen.

8. The compound of claim 1, wherein A is bromine.

9. The compound of claim 1, wherein A is iodine.

10. A pharmaceutical composition comprising as an active ingredient the compound of claim 1 and a pharmaceutical acceptable carrier.

11. The pharmaceutical composition of claim 10, packaged in a packaging material and identified in print, in or on said packaging material, for use in the treatment of an EGFR-tyrosine kinase related disease or disorder.

12. A method of treating an EGFR-tyrosine kinase related disease or disorder in a subject in need thereof, the method comprising administering to the subject a therapeutically effective amount of the pharmaceutical composition of claim 10.

13. The method of claim 12, wherein said EGFR-tyrosine kinase related disease or disorder is a cell proliferative disorder.

14. The method of claim 13, wherein said cell proliferative disorder is selected from the group consisting of papilloma, blastoglioma, Kaposi's sarcoma, melanoma, lung cancer, ovarian cancer, prostate cancer, squamous cell carcinoma, astrocytoma, head cancer, neck cancer, bladder cancer, breast cancer, lung cancer, colorectal cancer, thyroid cancer, pancreatic cancer, gastric cancer, hepatocellular carcinoma, leukemia, lymphoma, Hodgkin's disease and Burkitt's disease.

15. A method of inhibiting cell proliferation, the method comprising subjecting the cell to the compound of claim 1.

16. A method of synthesizing a compound having the general Formula II: 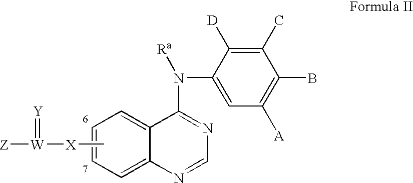
wherein:
X—W(═Y)-Z is at position 6 or 7 of the quinazoline ring;
X is selected from the group consisting of —NR¹—, —O—, —NH—NR¹—, —O—NR¹—, NH—CHR¹—, —CHR¹—NH—, —CHR¹—O—, —O—CHR¹—, —CHR¹—CH₂— and —CHR¹—S— or absent;
W is carbon;
Y is selected from the group consisting of oxygen and sulfur;
Z is —CR²R³R⁴;
R^{a} is selected from the group consisting of hydrogen or alkyl having 1-8 carbon atoms;
A, B, C and D are each independently selected from the group consisting of hydrogen and a non-radioactive derivatizing group;
R¹ is selected from the group consisting of hydrogen and substituted or non-substituted alkyl having 1-6 carbon atoms;
R² is a leaving group selected from the group consisting of halogen, alkoxy, aryloxy, thioalkoxy, thioaryloxy, azide, sulfinyl, sulfonyl, sulfonamide, phosphonyl, phosphinyl, carboxy and carbamyl; and
R³ and R⁴ are each independently selected from the group consisting of hydrogen and a second derivatizing group, the method comprising:
(a) coupling an aniline derivatized by said R^{a}, A, B, C and D with a 4-chloroquinazoline substituted at position 6 and/or 7 by at least one reactive group, so as to produce a reactive 4-(phenylamino)quinazoline derivatized by said A, B, C and D; and
(b) reacting said reactive 4-(phenylamino)quinazoline with a reactive carboxylic derivative substituted at the α position by said R², R³ and R⁴.

17. The method of claim 16, wherein said reactive 4-(phenylamino)quinazoline is 4-(phenylamino)-6-nitroquinazoline, the method further comprising, prior to step (b):
(c) reducing said 4-(phenylamino)-6-nitroquinazoline so as to produce a 4-(phenylamino)-6-aminoquinazoline derivatized by said A, B, C and D.

18. The method of claim 16, wherein said 4-chloroquinazoline is substituted at positions 6 and 7 by a first and a second reactive groups, the method further comprising, prior to step (b):
(d) reacting said reactive 4-(phenylamino)quinazoline with a chemically reactive group.

19. The method of claim 16, wherein said reactive carboxylic derivative is selected from the group consisting of α-chloroacetyl chloride and α-methoxyacetyl chloride.

20. A radiolabeled compound having the general Formula III: 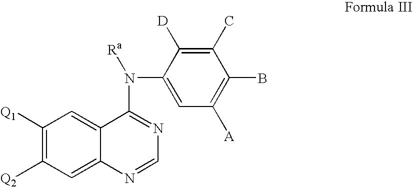
wherein:
Q1 is X—W(═Y)-Z and Q2 is selected from the group consisting of hydrogen, halogen, alkoxy, hydroxy, thiohydroxy, thioalkoxy, alkylamino and amino, or
Q1 is selected from the group consisting of hydrogen, halogen, alkoxy, hydroxy, thiohydroxy, thioalkoxy, alkylamino and amino and Q2 is X—W(═Y)-Z;
X is selected from the group consisting of —NR¹—, —O—, —NH—NR¹—, —O—NR¹—, NH—CHR¹—, —CHR¹—NH—, —CHR¹—O—, —O—CHR¹—, —CHR¹—CH₂— and —CHR¹—S— or absent;
W is carbon;
Y is selected from the group consisting of oxygen and sulfur;
Z is —CR²R³R⁴;
R^{a} is selected from the group consisting of hydrogen or alkyl having 1-8 carbon atoms;
A, B, C and D are each independently selected from the group consisting of hydrogen, a first non-radioactive derivatizing group and a first radioactive derivatizing group selected from a radioactive bromine, a radioactive iodine and a radioactive fluorine;
R¹ is selected from the group consisting of hydrogen, and substituted or non-substituted alkyl having 1-6 carbon atoms;
R² is a leaving group; and
R³ and R⁴ are each independently selected from the group consisting of hydrogen, a second non-radioactive derivatizing group and a second radioactive derivatizing group containing a radioactive carbon, a radioactive fluorine, a radioactive bromine and/or a radioactive iodine;
provided that the compound comprises at least one radioactive atom.

21. The radiolabeled compound of claim 20, wherein said leaving group is selected from the group consisting of halogen, alkoxy, aryloxy, thioalkoxy, thioaryloxy, azide, sulfinyl, sulfonyl, sulfonamide, phosphonyl, phosphinyl, carboxy and carbamyl.

22. The radiolabeled compound of claim 20, wherein Q1 is X—W(═Y)-Z and Q2 is selected from the group consisting of hydrogen, halogen, alkoxy, hydroxy, thiohydroxy, thioalkoxy, alkylamino and amino.

23. The radiolabeled compound of claim 20, wherein X is said —NR¹— and Y is oxygen.

24. The radiolabeled compound of claim 23, wherein each of R¹, R³ and R⁴ is hydrogen.

25. The radiolabeled compound of claim 20, wherein R² is a leaving group selected from the group consisting of alkoxy and halogen.

26. The radiolabeled compound of claim 20, wherein D is said radioactive fluorine.

27. The radiolabeled compound of claim 26, wherein A and B are each chlorine and C is hydrogen.

28. The radiolabeled compound of claim 20, wherein A is said radioactive bromine.

29. The radiolabeled compound of claim 20, wherein A is said radioactive iodine.

30. A pharmaceutical composition comprising as an active ingredient the radiolabeled compound of claim 20 and a pharmaceutical acceptable carrier.

31. A method of monitoring the level of epidermal growth factor receptor within a body of a patient, the method comprising:
(a) administering to the patient the radiolabeled compound of claim 20; and
(b) employing a nuclear imaging technique for monitoring a distribution of the compound within the body or within a portion thereof.

32. The method of claim 31, wherein said technique is positron emission tomography.

33. The method of claim 31, wherein said technique is single photon emission computed tomography.

34. A method of radiotherapy comprising administering to a patient a therapeutically effective amount of the radiolabeled compound of claim 20.

35. A method of synthesizing a radiolabeled compound having the general Formula IV: 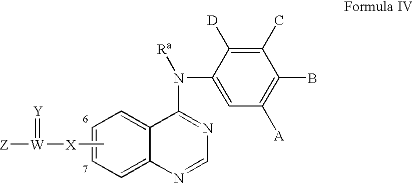
wherein:
X—W(═Y)-Z is at position 6 or 7 of the quinazoline ring;
X is selected from the group consisting of —NR¹—, —O—, —NH—NR¹—, —O—NR¹—, NH—CHR¹—, —CHR¹—NH—, —CHR¹—O—, —O—CHR¹—, —CHR¹—CH₂— and —CHR¹—S— or absent;
W is carbon;
Y is selected from the group consisting of oxygen and sulfur;
Z is —CR²R³R⁴;
R^{a} is selected from the group consisting of hydrogen or alkyl having 1-8 carbon atoms;
A, B, C and D are each independently selected from the group consisting of hydrogen, a first non-radioactive derivatizing group and a fluorine-18, provided that at least one of A, B, C and D is said fluorine-18;
R¹ is selected from the group consisting of hydrogen, and substituted or non-substituted alkyl having 1-6 carbon atoms;
R² is a leaving group; and
R³ and R⁴ are each independently selected from the group consisting of hydrogen and a second non-radioactive derivatizing group, the method comprising:
(a) providing a fluorine-18 labeled aniline derivatized by said R^{a}, A, B, C and D, wherein at least one of A, B, C and D is said fluorine-18;
(b) coupling said fluorine-18 labeled aniline derivatized by said Rₐ, A, B, C and D with 4-chloroquinazoline substituted at position 6 and/or 7 by at least one reactive group, so as to produce a reactive fluorine-18 labeled 4-(phenylamino)quinazoline derivatized by said A, B, C and D; and
(c) reacting said reactive fluorine-18 labeled 4-(phenylamino)quinazoline with a reactive carboxylic derivative substituted at the α position by said R², R³ and R⁴.

36. The method of claim 35, wherein said leaving group is selected from the group consisting of halogen, alkoxy, aryloxy, thioalkoxy, thioaryloxy, azide, sulfinyl, sulfonyl, sulfonamide, phosphonyl, phosphinyl, carboxy and carbamyl.

37. The method of claim 35, wherein said reactive fluorine-18 labeled 4-(phenylamino)quinazoline is fluorine-18 labeled 4-(phenylamino)-6-nitroquinazoline, the method further comprising, prior to step (c):
(d) reducing said fluorine-18 labeled 4-(phenylamino)-6-nitroquinazoline, so as to produce a fluorine-18 labeled 4-(phenylamino)-6-aminoquinazoline derivatized by said A, B, C and D.

38. The method of claim 35, wherein said 4-chloroquinazoline is substituted at positions 6 and 7 by a first and a second reactive groups, the method further comprising, prior to step (c):
(e) reacting said reactive fluorine-18 labeled 4-(phenylamino)quinazoline with a chemically reactive group.

39. The method of claim 35, wherein said reactive carboxylic derivative is selected from the group consisting of α-chloroacetyl chloride and α-methoxyacetyl chloride.

40. A method of synthesizing a radiolabeled compound having the general Formula V: 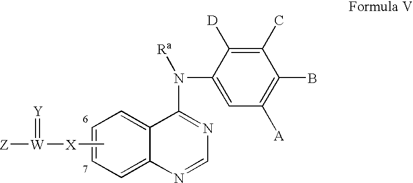
wherein:
X—W(═Y)-Z is at position 6 or 7 of the quinazoline ring;
X is selected from the group consisting of —NR¹—, —O—, —NH—NR¹—, —O—NR—, NH—CHR¹—, —CHR¹—NH—, —CHR¹—O—, —O—CHR¹—, —CHR¹—CH₂— and —CHR¹—S— or absent;
W is a non-radioactive carbon;
Y is selected from the group consisting of oxygen and sulfur;
Z is —CR²R³R⁴;
R^{a} is selected from the group consisting of hydrogen or alkyl having 1-8 carbon atoms;
A, B, C and D are each independently selected from the group consisting of hydrogen, a first non-radioactive derivatizing group and a radioactive atom selected from a radioactive bromine and a radioactive iodine, provided that at least one of A, B, C and D is said radioactive bromine or said radioactive iodine;
R¹ is selected from the group consisting of hydrogen, and substituted or non-substituted alkyl having 1-6 carbon atoms;
R² is a leaving group; and
R³ and R⁴ are each independently selected from the group consisting of hydrogen and a second non-radioactive derivatizing group, the method comprising:
(a) coupling an aniline derivatized by said R^{a}, A, B, C and D, wherein at least one of A, B, C and D is a halogen, with a 4-chloroquinazoline substituted at position 6 and/or 7 by at least one reactive group, so as to produce a reactive 4-(phenylamino)quinazoline derivatized by said A, B, C and D, wherein at least one of A, B, C and D is said halogen;
(b) radiolabeling said reactive 4-(phenylamino)quinazoline derivatized by said A, B, C and D with a radioactive bromine or a radioactive iodine, so as to produce a radioactive bromine labeled or a radioactive iodine labeled reactive 4-(phenylamino)quinazoline derivatized by said A, B, C and D, wherein at least one of said A, B, C and D is said radioactive bromine or said radioactive iodine; and
(c) reacting said radioactive bromine labeled or radioactive iodine labeled reactive 4-(phenylamino)quinazoline with a reactive carboxylic derivative substituted at the α position by said R², R³ and R⁴.

41. The method of claim 40, wherein said leaving group is selected from the group consisting of halogen, alkoxy, aryloxy, thioalkoxy, thioaryloxy, azide, sulfinyl, sulfonyl, sulfonamide, phosphonyl, phosphinyl, Carboxy and Carbamyl.

42. The method of claim 40, wherein said reactive 4-(phenylamino)quinazoline is 4-(phenylamino)-6-nitroquinazoline, the method further comprising, prior to step (b):
(d) reducing said 4-(phenylamino)-6-nitroquinazoline, so as to produce a 4-(phenylamino)-6-aminoquinazoline derivatized by said A, B, C and D, wherein at least one of said A, B, C and D is said halogen.

43. The method of claim 40, wherein said 4-chloroquinazoline is substituted at positions 6 and 7 by a first and a second reactive groups, the method further comprising, prior to step (c):
(e) reacting said reactive radioactive bromine labeled or radioactive iodine labeled 4-(phenylamino)quinazoline with a chemically reactive group.

44. The method of claim 40, wherein said reactive carboxylic derivative is selected from the group consisting of α-chloroacetyl chloride and α-methoxyacetyl chloride.

45. A method of synthesizing a radiolabeled compound having the general Formula IV: 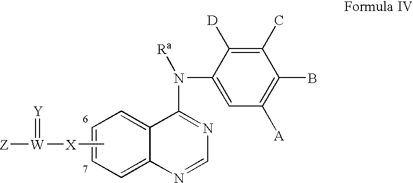
wherein:
X—W(═Y)-Z is at position 6 or 7 of the quinazoline ring;
X is selected from the group consisting —NR¹—, —O—, —NH—NR¹—, —O—NR¹—, NH—CHR¹—, —CHR¹—NH—, —CHR¹—O—, —O—CHR¹—, —CHR¹—CH₂— and —CHR¹—S— or absent;
W is carbon;
Y is selected from the group consisting of oxygen and sulfur;
Z —CR²R³R⁴;
R^{a} is selected from the group consisting of hydrogen or alkyl having 1-8 carbon atoms;
A, B, C and D are each independently selected from the group consisting of hydrogen, a non-radioactive derivatizing group and a fluorine-18, provided that at least one of A, B, C and D is said fluorine-18;
R¹ is selected from the group consisting of hydrogen, and substituted or non-substituted alkyl having 1-6 carbon atoms;
R² is a leaving group; and
R³ and R⁴ are each independently selected from the group consisting of hydrogen and a second non-radioactive derivatizing group, the method comprising:
(a) coupling an aniline derivatized by amine, by said R^{a}, and by three of said A, B, C and D which are not said fluorine-18, with a 4-chloroquinazoline substituted at position 6 or 7 by a first reactive group, so as to produce a reactive 4-(amino-substituted phenylamino) quinazoline derivatized by said amine, said R^{a}, and three of said A, B, C and D which are not said fluorine-18;
(b) converting said reactive 4-(amino-substituted phenylamino)quinazoline derivatized by said amine, said R^{a}, and three of said A, B, C and D which are not said fluorine-18 into a quaternary ammonium salt thereof,
(c) reacting said quaternary ammonium salt with a fluorine-18 labeled ion, so as to produce a reactive fluorine-18 labeled 4-(phenylamino)quinazoline derivatized by said Ra, A, B, C and D; and
(d) reacting said reactive fluorine-18 labeled 4-(phenylamino)quinazoline with a reactive carboxylic derivative substituted at the a position by said R², R³ and R⁴.

46. The method of claim 45, wherein said leaving group is selected from the group consisting of halogen, alkoxy, aryloxy, thioalkoxy, thioaryloxy, azide, sulfinyl, sulfonyl, sulfonamide, phosphonyl, phosphinyl. Carboxy and Carbamyl.

47. The method of claim 45, wherein said reactive fluorine-18 labeled 4-(phenylamino)quinazoline is fluorine-18 labeled 4-(phenylamino)-6-nitroquinazoline, the method further comprising, prior to step (d):
(e) reducing said fluorine-18 labeled 4-(phenylamino)-6-nitroquinazoline, so as to produce a fluorine-18 labeled 4-(phenylamino)-6-aminoquinazoline derivatized by said A, B, C and D.

48. The method of claim 45, wherein said 4-chloroquinazoline is substituted at positions 6 and 7 by a first and a second reactive groups, the method further comprising, prior to step (d):
(f) reacting said reactive fluorine-18 labeled 4-(phenylamino)quinazoline with a chemically reactive group.

49. The method of claim 45, wherein said reactive carboxylic derivative is selected from the group consisting of α-chloroacetyl chloride and α-methoxyacetyl chloride.

50. A method of synthesizing a radiolabeled compound having the general Formula VI: 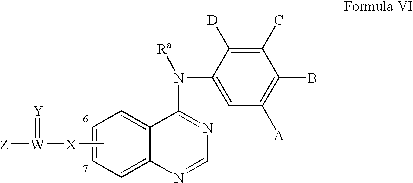
wherein:
X—W(═Y)-Z is at position 6 or 7 of the quinazoline ring;
X is selected from the group consisting of —NR¹—, —O—, —NH—NR¹—, —O—NR¹—, NH—CHR¹—, —CHR¹—NH—, —CHR¹—O—, —O—CHR¹—, —CHR¹—CH₂— and —CHR¹—S— or absent;
W is carbon;
Y is selected from the group consisting of oxygen and sulfur;
Z is —CR²R³R⁴;
R^{a} is selected from the group consisting of hydrogen or alkyl having 1-8 carbon atoms;
A, B, C and D are each independently selected from the group consisting of hydrogen and a first non-radioactive derivatizing group;
R¹ is selected from the group consisting of hydrogen and substituted or non-substituted alkyl having 1-6 carbon atoms;
R² is a leaving group; and
R³ and R⁴ are each independently selected from the group consisting of hydrogen, a second non-radioactive derivatizing group and a second radioactive derivatizing group containing a radioactive fluorine, a radioactive bromine, a radioactive iodine and/or a radioactive iodine, the method comprising:
(a) coupling an aniline derivatized by said R^{a}, A, B, C and D with a 4-chloroquinazoline substituted at position 6 and/or 7 by at least one reactive group, so as to produce a reactive 4-(phenylamino)quinazoline derivatized by said A, B, C and D; and
(b) reacting said reactive 4-(phenylamino)quinazoline with a radiolabeled reactive carboxylic derivative substituted at the α position by said R², R³ and R⁴.

51. The method of claim 50, wherein said leaving group is selected from the group consisting of halogen, alkoxy, aryloxy, thioalkoxy, thioaryloxy, azide, sulfinyl, sulfonyl, sulfonamide, phosphonyl, phosphinyl, Carboxy and Carbamyl.

52. The method of claim 50, wherein said reactive 4-(phenylamino)quinazoline is 4-(phenylamino)-6-nitroquinazoline, the method further comprising, prior to step (b):
(c) reducing said 4-(phenylamino)-6-nitroquinazoline so as to produce a 4-(phenylamino)-6-aminoquinazoline derivatized by said A, B, C and D.

53. The method of claim 50, wherein said 4-chloroquinazoline is substituted at positions 6 and 7 by a first and a second reactive groups, the method further comprising, prior to step (b):
(d) reacting said reactive 4-(phenylamino)quinazoline with a chemically reactive group.
